(19) United States
(12) Patent Application Publication
(10) Pub. No.: US 2021/0147481 A1
(43) Pub. Date: May 20, 2021

(54) PEPTIDE-BASED INHIBITORS OF MARK FAMILY PROTEINS

(71) Applicant: Ohio Universtiy, Athens, OH (US)

(72) Inventors: Justin M. Holub, Athens, OH (US); Robert A. Colvin, Athens, OH (US)

(73) Assignee: Ohio University, Athens, OH (US)

(21) Appl. No.: 16/605,302

(22) PCT Filed: Apr. 20, 2018
(86) PCT No.: PCT/US2018/028481

§ 371 Date: Oct. 15, 2019

Related U.S. Application Data
(60) Provisional application No. 62/488,134, filed on Apr. 21, 2017.

(51) Int. Cl. C07K 7/08; A61P 25/28

(52) U.S. Cl. CPC C07K 7/08; A61P 25/28

(57) ABSTRACT

Compositions and methods for the inhibiting tau phosphorylation, and treating or preventing neurodegenerative diseases, utilizing a tR1 peptide having the amino acid sequence of NVKSKIGSTENLK [SEQ ID NO: 1], or a variant thereof, are described.

### RELATED APPLICATIONS

This application claims priority to U.S. Provisional Application Ser. No. 62/488,134 filed under 35 U.S.C. § 111(b) on Apr. 21, 2017, the disclosure of which is incorporated herein by reference in its entirety.

### STATEMENT REGARDING FEDERALLY SPONSORED RESEARCH

This invention was made with no government support. The government has no rights in this invention.

### SEQUENCE LISTING

The instant application contains a Sequence Listing which has been submitted electronically in ASCII format and is hereby incorporated by reference in its entirety. Said ASCII copy, created on Apr. 11, 2018, is named 59232-PCT_OU-17027_SL.txt and is 987 bytes in size.

### BACKGROUND OF THE INVENTION

Protein kinases play critical roles in regulating cellular function by controlling the phosphorylation state of proteins. The phosphorylation state of a protein can have profound effects on its regulation, activity, trafficking, and ability to interact with other biomolecules. Protein kinases are therefore considered crucial regulators of cellular metabolism, signal transduction, protein activation, cellular transport, and secretory processes. The human protein kinase superfamily is composed of approximately 500 genes, constituting about 2% of the entire human genome. Furthermore, up to 30% of all human proteins are thought to be modified though some kinase activity. Due to their extensive influence in regulating processes that control cell growth, differentiation, and metabolism, aberrations in protein kinase activity can result in the onset of diseases including diabetes, inflammation, cancer, and neurodegenerative disorders. Consequently, protein kinases are targets for therapeutics.

Proteins in the microtubule affinity-regulating kinase (MARK) family are important for establishing and maintaining the structural integrity of microtubules. Dysregulation of MARK function has been linked to cancer, inflammation, and neurodegenerative disorders such as Alzheimer's disease (AD). In neurons, MARK2 proteins phosphlorylate the microtubule-associated protein tau, a protein involved in the assembly, maintenance, and stability of microtubules. Phosphorylation of tau causes it to disengage from microtubules, leading to loss in microtubule stability. Hyper-phosphorylated tau proteins can aggregate in to higher order filaments, leading to neurodegenerative disorders such as AD and frontotemporal dementia.

Tau stabilizes microtubules by interacting with tubulins in neurons. tau that is post-translationally phosphorylated can dissociate from tubulin, leaving polymeric microtubule structures in highly dynamic states. Tau phosphorylation at specific amino acids is precisely regulated by tau kinases and tau phosphatases. When this machinery is dysregulated, tau becomes hyper-phosphorylated. (FIG. 11.) Prolonged dissociation of hyper-phosphorylated tau induces microtubule collapse and disrupts axonal transport. Phosphorylated tau can form prion-like oligomers, which have been impliaced in the pathogensis of neurodegenerative disease such as AD. Elevated activity of tau kinases, including the MARK2, has been demonstrated in AD.

It would be desirable to develop a highly selective inhibitor of MARK2 function.

### SUMMARY OF THE INVENTION

Provided is a composition comprising peptide (designated tR1) consisting of the amino acid sequence NVKSKIGSTENLK [SEQ ID NO: 1], or a variant thereof. In certain embodiments, the variant has at least 61% sequence identity to the amino acid sequence of tR1. In certain embodiments, the variant has at least 69% sequence identity to the amino acid sequence of tR1. In certain embodiments, the variant has at least 76% sequence identity to the amino acid sequence of tR1. In certain embodiments, the variant has at least 84% sequence identity to the amino acid sequence of tR1. In certain embodiments, the variant has at least 92% sequence identity to the amino acid sequence of tR1. In certain embodiments, the composition further includes a pharmaceutically acceptable excipient, diluent, adjuvant, or carrier.

Further provided is a method of inhibiting MARK2 function in a subject, the method comprising administering to a subject an effective amount of a tR1 peptide consisting of the amino acid sequence NVKSKIGSTENLK [SEQ ID NO: 1], or a variant thereof, to inhibit MARK2 function in the subject. In certain embodiments, the variant has at least 61% sequence identity to the amino acid sequence of tR1. In certain embodiments, the variant has at least 69% sequence identity to the amino acid sequence of tR1. In certain embodiments, the variant has at least 76% sequence identity to the amino acid sequence of tR1. In certain embodiments, the variant has at least 84% sequence identity to the amino acid sequence of tR1. In certain embodiments, the variant has at least 92% sequence identity to the amino acid sequence of tR1.

Further provided is a method of inhibiting phosphorylation of tau Ser262 in primary cortical neurons, the method comprising administering to primary cortical neurons an effective amount of a tR1 peptide consisting of the amino acid sequence NVKSKIGSTENLK [SEQ ID NO: 1], or a variant thereof, to inhibit phosphorylation of tau Ser262 in primary cortical neurons. In certain embodiments, the tR1 peptide does not inhibit GSK-3β-mediated phosphorylation of tau Thr231 in the primary cortical neurons. In certain embodiments, the variant has at least 61% sequence identity to the amino acid sequence of tR1. In certain embodiments, the variant has at least 69% sequence identity to the amino acid sequence of tR1. In certain embodiments, the variant has at least 76% sequence identity to the amino acid sequence of tR1. In certain embodiments, the variant has at least 84% sequence identity to the amino acid sequence of tR1. In certain embodiments, the variant has at least 92% sequence identity to the amino acid sequence of tR1.

Further provided is a method of treating, preventing, or ameliorating a neurodegenerative disease, the method comprising administering to a subject in need thereof an effective amount of a tR1 peptide consisting of the amino acid sequence NVKSKIGSTENLK [SEQ ID NO: 1], or a variant thereof, to treat, prevent, or ameliorate a neurodegenerative disease in the subject. In certain embodiments, the neurodegenerative disease is AD or frontotemporal dementia. In certain embodiments, the tR1 peptide does not inhibit GSK-3β-mediated phosphorylation of tau Thr231 in the primary cortical neurons. In certain embodiments, the variant has at least 61% sequence identity to the amino acid sequence of tR1. In certain embodiments, the variant has at least 69% sequence identity to the amino acid sequence of tR1. In certain embodiments, the variant has at least 76% sequence identity to the amino acid sequence of tR1. In certain embodiments, the variant has at least 84% sequence identity to the amino acid sequence of tR1. In certain embodiments, the variant has at least 92% sequence identity to the amino acid sequence of tR1.

Further provided is a method of inhibiting a MARK family protein, the method comprising administering to a subject an effective amount of a tR1 peptide consisting of the amino acid sequence NVKSKIGSTENLK [SEQ ID NO: 1], or a variant thereof, to inhibit a MARK family protein in the subject. In certain embodiments, the variant has at least 61% sequence identity to the amino acid sequence of tR1. In certain embodiments, the variant has at least 69% sequence identity to the amino acid sequence of tR1. In certain embodiments, the variant has at least 76% sequence identity to the amino acid sequence of tR1. In certain embodiments, the variant has at least 84% sequence identity to the amino acid sequence of tR1. In certain embodiments, the variant has at least 92% sequence identity to the amino acid sequence of tR1.

Further provided is a use of a synthetic peptide to inhibit MARK2-mediated tau phosphorylation in vitro. In certain embodiments, the synthetic peptide consists of the amino acid sequence NVKSKIGSTENLK [SEQ ID NO: 1], or a variant thereof. In certain embodiments, the variant has at least 61% sequence identity to the amino acid sequence of tR1. In certain embodiments, the variant has at least 69% sequence identity to the amino acid sequence of tR1. In certain embodiments, the variant has at least 76% sequence identity to the amino acid sequence of tR1. In certain embodiments, the variant has at least 84% sequence identity to the amino acid sequence of tR1. In certain embodiments, the variant has at least 92% sequence identity to the amino acid sequence of tR1. In certain embodiments, the synthetic peptide mimics the tau R1 repeat domain.

Further provided is a use of a synthetic peptide to inhibit a MARK family protein. In certain embodiments, the synthetic peptide consists of the amino acid sequence NVKSKIGSTENLK [SEQ ID NO: 1], or a variant thereof. In certain embodiments, the variant has at least 61% sequence identity to the amino acid sequence of tR1. In certain embodiments, the variant has at least 69% sequence identity to the amino acid sequence of tR1. In certain embodiments, the variant has at least 76% sequence identity to the amino acid sequence of tR1. In certain embodiments, the variant has at least 84% sequence identity to the amino acid sequence of tR1. In certain embodiments, the variant has at least 92% sequence identity to the amino acid sequence of tR1. In certain embodiments, the synthetic peptide mimics the tau R1 repeat domain.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIGS. 1A-1E: The tR1 peptide inhibits MARK2-mediated tau phosphorylation in vitro:

FIG. 1A shows bar diagrams of hTau and hTau-derived constructs used in the examples herein. The top bar shows the full-length hTau protein displaying projection and microtubule-binding domains. N1 and N2 indicate N-terminal insert sequences; P1 and P2 are proline-rich segments. The lower bar shows the four-repeat domain hTau-K18 protein and tR1 peptide [SEQ ID NO: 1] derived from the hTau R1 sequence. The tR1 Ser residue phosphorylated by MARK2 is shown in red.

FIG. 1B: Western blot analysis demonstrating tR1 peptides inhibit MARK2-mediated phosphorylation of hTau-K18 proteins. Concentrations (μM) of tR1 peptide are shown above each lane. Upper image shows western blot using α-Tau pSer262 antibody; lower image shows western blot using α-Tau (total-tau) antibody. Molecular weight markers (kD) are shown to the left of western blot images.

FIG. 1C: Percent inhibition of MARK2-mediated hTau-K18 Ser262 phosphorylation by tR1 peptides as quantified from western blot experiments. Columns represent average of three separate experiments; error bars are standard deviation. Statistical analyses were performed using Student's t-test comparing percent inhibition of treated samples to that of untreated (0 αM tR1) controls; *, P<0.05

FIG. 1D: Bar graph showing the densitometric quantifications of the three lanes shown in FIG. 1C. Every hTau-K18 pS262 band is normalized to the total hTau-K18 band. Ratios are plotted in the bar graph.

FIG. 1E shows a scheme utilizing tR1 peptide as a competitive inhibitor of MARK2-dependent phosphorylation of endogenous tau.

FIGS. 2A-2D: The tR1 peptide is internalized by cultured primary neurons:

FIG. 2A shows fluorescently labeled tR1 peptides are internalized by rat primary cortical neurons and accumulate in vesicular structures. The left image shows a confocal micrograph of untreated neurons; the middle image shows a confocal micrograph of neurons treated with 10 μM tR1, where arrowheads indicate fluorescent puncta within the cell soma and neurites; and the right image shows a confocal micrograph of neurons treated with 10 μM tR1 and 10 mM sodium azide (NaN₃). DAPI stain (blue) shows location of cell nuclei. Scale bar: 10 μm.

FIG. 2B shows disruption of endosomes facilitates the dispersal of tR1 peptides in rat primary cortical neurons. The left image shows a confocal micrograph of cells co-treated with 10 μM ^{Flu}tR1 and 50 nM bafilomycin (Baf); the middle image shows a confocal micrograph of cells co-treated with 10 μM ^{Flu}tR1 and 100 μM chloroquine (CLQ); and the right image shows a confocal micrograph of cells co-treated with 10 μM ^{Flu}tR1 and 5 μM phenylarsine oxide (PAO.

FIG. 2C shows a particle analysis of the images in FIG. 2B.

FIG. 2D shows a diagram of endosomal internalization of tR1 peptides and the competition between synthetic tR1 peptides and the endogenous tau proteins for MARK2 binding.

FIGS. 3A-3B: tR1 peptides inhibit phosphorylation of tau Ser262 in rat primary cortical neurons:

FIG. 3A shows immunofluorescence images demonstrating the inhibition of MARK2-dependent tau phosphorylation of Ser262 by tR1 peptides in cultured neurons. Top row: cells untreated or exposed to 5 μM phenylarsine oxide (PAO) for 45 min with or without 30 μM tR1 peptides. Middle row: cells co-treated with PAO and 50 nM bafilomycin A1 (Baf) in the presence of 0, 10, or 30 μM tR1 peptide. Bottom row: cells co-treated with PAO and 100 μM chloroquine (CLQ) in the presence of 0, 10, or 30 μM tR1 peptide. Insets show similarly treated cells that showed weaker inhibition of MARK2-mediated phosphorylation of tau Ser262. Scale bar is 10 μm.

FIG. 3B shows a scatter plot displaying mean fluorescent intensities of soma areas in fluorescent micrographs shown in FIG. 3A. Each data point represents the background-subtracted mean fluorescent intensity of a single image. Statistical analyses were performed using one-way ANOVA and Tukey's multiple comparison test. Mean±SEM; *, P<0.05; ns—not significant.

FIGS. 4A-4B: tR1 peptides do not inhibit phosplorylation of tau Thr231 in rat primary cortical neurons:

FIG. 4A shows immunofluorescence images showing that tR1 peptides neither inhibit the baseline tau phosphorylation of Thr231 nor reverse the PAO-induced decrease of tau phosphorylation of Thr231 in cultured primary cortical neurons.

FIG. 4B shows a scatter plot displaying mean fluorescence intensities of soma areas in fluorescent micrographs showing in FIG. 4A. One-way ANOVA and Tukey's multiple comparison test. *, P<0.5; ns—not significant.

FIGS. 5A-5D: Purification and characterization of recombinant MARK2 and hTau-K18 proteins:

FIG. 5A shows purification and characterization of MARK2 proteins. The left image shows SDS-PAGE of MARK2 purification by column chromatography; the size marker (kD) is shown to the left of the gel image. M: size marker, FT: flow through. W1-3: column washes: E1-5 column elutions. Arrows indicate the position of full-length MARK2 and truncated isoforms within the gel. The right image displays bar diagrams of potential truncated products of MARK2; size of each isoform (kD) is indicated to the left of each bar diagram.

FIG. 5B shows purification of hTau-K18 proteins. The image shows SDS-PAGE of hTau-K18 purification by column chromatography; the size marker (kD) is shown to the left of the gel image. M: size marker, FT: flow through, W1-3: column washes: E1-5 column elutions. Arrow indicates the position of hTau-K18 within the gel.

FIG. 5C shows wavelength-dependent circular dichroism (CD) spectra of MARK2 (5 μM in phosphorylation buffer) at 25° C. The inset shows a thermal scan of MARK2 (5 μM in phosphorylation buffer) at 222 nm: Tₘ=61.1° C.

FIG. 5D shows wavelength-dependent CD spectra of hTau-K18 (10 μM in phosphorylation buffer) at 25° C. All CD spectra represent background-subtracted (buffer only) average of four scans.

FIGS. 6A-6D: Validation of tR1 as a substrate for MARK2:

FIG. 6A shows the primary sequence of tR1 peptides used in the examples herein. The tR1 peptide is acylated at its N-terminus (Ac); ^{Flu}tR1 is labeled on its N-terminus with 5-carboxyfluorescein (Flu); phosphorylated Ser residue, corresponding to Ser262 in full-length hTau, is shown underlined in red. “NVKSKIGSTENLK” is disclosed as [SEQ ID NO: 1].

FIG. 6B shows a western blot analysis showing that the tR1 peptide is a substrate for MARK2. Varying concentrations of phosphorylated tR1 peptide (0-100 μM) were loaded onto the gel and the blot was developed using α-pTau Ser262 antibody. Size marker (kD) is shown to the left of the image.

FIG. 6C shows a schematic representation of antibody-linked fluorescence polarization binding assay used to evaluate phosphorylation of tR1 by MARK2.

FIG. 6D shows a column graph displaying results from the antibody-linked fluorescence polarization binding assay.

FIG. 7A: Effect of tR1 peptides on the viability of rat primary cortical neurons. Bar graph shows the results of colorimetric MTT assay testing the effects of tR1 peptides on the viability of rat primary cortical neurons. Cells were grown in the presence of varying concentrations of tR1 peptide for 72 hours. Cell viability was then measured using an MTT assay kit according to the manufacturer's instructions. Relative cell viability was quantified as a measure of control (untreated) cells.

FIG. 7B: Immunofluorescence micrographs of rat primary cortical neurons utilized for western blot. Neurons were plated at high density onto coated 6-well culture plates and were grown 5-7 DIV; each well contained a single poly-E-coated coverslip. Cultured neurons were treated as described and fixed using the procedures used for western blot analysis. Following fixation, the coverslips were processed using identical immunofluorescence procedures. DAPI staining (blue) was used to visualize the cell nuclei. Secondary antibodies tagged with Alexa-488 fluorophore were used to target the primary antibodies targeting tau pSer262. Scale bar is 10 μm.

FIGS. 8A-8B: Characterization of tR1 peptides by analytical HPLC and mass spectrometry:

FIG. 8A shows analytical HPLC chromatograms of peptides used in the examples herein. All spectra were monitored at 214 nm; AU: normalized absorbance units.

FIG. 8B shows deconvoluted ESI mass spectra of peptides used in the examples herein. Raw data was deconvoluted to remove multiply charged ions from the final mass spectrum.

FIG. 9: Table 1, displaying sequences and mass data of acyl- and fluorescently-labeled tR1 peptides used in the examples herein. The Serine residue corresponding to Ser262 of full-length hTau is shown in red in both peptide sequences. “NVKSKIGSTENLK” is disclosed as [SEQ ID NO: 1].

FIG. 10: Diagram illustrating that some embodiments of synthetic tR1 peptides may perform better endosomal escape.

FIG. 11: Illustration showing dysregulation of tau phosphorylation.

FIGS. 12A-12D: In vitro stability of tR1 peptides:

FIG. 12A: Stability of tR1 peptide in digestion buffer supplemented with 10 ng/μL trypsin as analyzed by analytical RP-HPLC. Lower spectrum shows HPLC chromatogram of tR1 peptide at 0 minutes incubation with trypsin; upper spectrum shows HPLC chromatogram of tR1 peptide after 15 minutes incubation with trypsin. Upper spectra offset: 300 nm. AU: absorbance units at 214 nm.

FIG. 12B: Stability of tR1 peptide in RPMI media supplemented with 25% (v/v) human serum as analyzed by analytical RP-HPLC. Peptide incubation times are shown to the right of each chromatogram.

FIG. 12C: Stability of tR1 peptide in NB media as analyzed by analytical RP-HPLC. Peptide incubation times are shown to the right of each chromatogram.

FIG. 12D: Quantification of peptide stability (fraction of intact tR1) as determined from chromatograms shown in panels b, and c. The fraction of intact tR1 was obtained as described in the Materials and Methods section. Circles: RPMI media supplemented with human serum; Squares: NB media.

### DETAILED DESCRIPTION OF THE INVENTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Provided herein is a synthetic peptide mimic (tR1) of the tau R1 repeat domain. The tR1 peptide is a cell-permeable ligand to selectively inhibit MARK2-mediated phosphorylation of endogenous tau. The tR1 peptide has the amino acid sequence of NVKSKIGSTENLK [SEQ ID NO: 1]. The term “tR1 peptides” is used herein to encompass the tR1 peptide having SEQ ID NO: 1, as well as variants and derivatives of this sequence which share the activity of the tR1 peptide having SEQ ID NO: 1, and the amino acid sequence of SEQ ID NO: 1 with one or more tags or labels, such as a fluorescent tag. Thus, in some embodiments, a tR1 peptide has an amino acid sequence consisting of NVKSKIGSTENLK [SEQ ID NO: 1]. In other embodiments, a tR1 peptide is a variant that has an amino acid sequence where one or more of the amino acids in SEQ ID NO: 1 has been substituted with another amino acid, but the peptide nonetheless has the ability to selectively inhibit MARK2-mediated phosphorylation of endogenous tau. Modifications to the tR1 sequence can be introduced by, for example, mutagenesis or protein synthesis. Such modifications include, for example, deletions from, insertions into, and/or substitutions within the amino acid sequence of tR1. Any combination of deletion, insertion, and substitution can be made to arrive at the final amino acid construct of the variant, provided that the final construct possesses the desired biological activity. Accordingly, in some embodiments, the variants have at least 61%, at least 69%, at least 76%, at least 84%, or at least 92% sequence identity to the amino acid sequence of tR1. Reference to a “% sequence identity” with respect to a reference polypeptide is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity.

In vitro assays have shown that tR1 peptides inhibit MARK2-mediated phosphorylation of tau at Ser262 within its R1 domain. Fluorescently-labeled tR1 (^{Flu}tR1) was internalized by primary cortical neurons and was present throughout the cell body, suggesting internalization by endocytosis. Furthermore, it has been shown that tR1 peptides inhibit the phenylarsine oxide (PAO)-mediated activation of MARK2 in primary rat cortical neurons. PAO upregulated MARK2 activity and increased endogenous tau phosphorylation of Ser262 significantly over untreated controls. Co-treatment of PAO-treated primary neurons with the tR1 peptide and endosomal disruptors bafilomycin A1 or chloroquine reduced MARK2-dependent tau phosphorylation of Ser262 partially but significantly. Notably, co-treatment of neurons with tR1 and chloroquine did not change the level of phosphorylated tau at Thr231. Taken together, these data indicate that tR1 peptides are internalized by cortical neurons and inhibit MARK2-dependent phosphorylation of endogenous tau at Ser262 when delivered to the cytosol. These results show that the synthetic tR1 peptide mimetics are capable of selectively inhibiting tau phosphorylation at kinase specific sites. Syntheic R domain peptides are therefore useful in peptide-based therapeutic development for neurodegenerative diseases.

The inhibitory effects of tR1 were found to be specific for MARK2, as this peptide did not interfere with the activity of other kinases along this pathway, including kinases that phosphorylate tau at Thr231. Importantly, the tR1 peptide represents a peptide-based kinase inhibitor that may serve to inhibit the MARK2-mediated hyper-phosphorylation of tau and may be used as a tool to dissect the complex nature of MARK2 biology.

The ability to selectively target and inhibit protein kinase activity represents a significant challenge for modern medicinal chemists. Inhibition strategies have included directly blocking phosphorylation activity through competitive inhibition at the kinase active site, clearing the dysfunctional kinase through antibody-mediated degradation, or designing inhibitors of kinase-protein interactions. To date, the vast majority of protein kinase inhibitors that have been developed are classified as small molecules that competitively target the ATP-binding pocket of the kinase catalytic domain. Due to a high degree of structural homology among catalytic domains of protein kinases, many small-molecule kinase inhibitors suffer from limited target selectivity. Furthermore, patients administered with small molecule kinase inhibitors often develop resistance and suffer relapse of the disease being treated. An alternative strategy to small-molecule inhibitors of protein kinases includes developing therapeutic antibodies designed to specifically target the dysregulated kinase. Antibodies are advantageous because they can be developed against discrete protein kinases by targeting epitopes on the protein surface, thus achieving a high level of target specificity. However, antibodies are not cell-permeable and many kinases lack extracellular domains that can be targeted using this method. Another approach that has emerged for targeting protein kinases includes the development of peptide-based inhibitors of kinase activity. These molecules are designed to mimic the canonical recognition motif of protein kinase substrates, and often include specific amino acids surrounding the phosphorylation site that are critical for substrate recognition by the target protein kinase. Some peptides that mimic discrete recognition motifs have the ability to act as competitive inhibitors of the target protein kinase and may provide a high degree of specificity in targeting select protein kinases with enhanced precision.

The microtubule affinity regulating kinases (MARK) are a family of Ser/Thr kinases that are involved in establishing cellular polarity and maintaining the structural integrity of the cytoskeleton. There are currently four known isoforms of MARK in humans (MARK1-4), each of which contains a similar catalytic domain Targets of the MARK protein kinase family include the microtubule-associated proteins (MAPs) MAP2, MAP4, and tau. A prominent target of the MARK2 isoform is the microtubule binding domain of human tau (hTau), which is phosphorylated within four KXGS [SEQ ID. NO: 2] recognition motifs located in four repeat (R) domains. Phosphorylation within the R domains of hTau decreases its affinity for microtubules, resulting in diminished stability of microtubule superstructure. While a degree of microtubule destabilization is necessary to facilitate neuronal polarization and neurite outgrowth, excess destabilization can result in necrosis and cell death. Consequently, detrimental effects to the cell are observed when the balance of hTau phosphorylation and de-phosphorylation becomes dysregulated.

Moreover, hyper-phosphorylated hTau can undergo mis-sorting and aggregation in neurons, leading to neurofibrillary tangles that can disrupt the structural integrity of neuronal networks. Early stage hTau hyper-phosphorylation by elevated physiological levels of MARK2 has become a hallmark of such debilitating diseases as AD and frontotemporal dementia. While it is known that the catalytic domain of MARK2 is activated by phosphorylation of Thr-208 by MARKK/TAO1 or LKB1, it is currently unclear what causes the over-activation of this kinase. Efforts have been initiated to develop small molecule inhibitors of MARK family kinases, which have culminated in the small-molecule MARK/Par-1 inhibitor 39621. However, it has been documented that 39621 targets all known MARK isoforms with similar affinity and its cross-reactivity with other protein kinases has not been fully characterized. It is believed that 39621 remains the only commercially available small-molecule inhibitor of MARK activity.

As the examples herein demonstrate, the tR1 peptides can be phosphorylated by MARK2 in the presence of ATP and this phosphorylation event can be inhibited by tR1 in a concentration-dependent manner. The tR1 peptides are internalized by primary cortical neurons via endocytosis. The tR1 peptides do not show toxicity to the primary cortical neurons. Once being delivered into the cytosol, tR1 peptides can selectively inhibit tau phosphorylation at Ser262, whereas tR1 peptides do not inhibit tau phosphorylation at Thr231.

The tR1 peptides can be formulated in a suitable pharmaceutical composition comprising at least one tR1 peptide and one or more pharmaceutically acceptable excipients, diluents, carriers, or adjuvants. Such compositions may further comprise one or more additional active agents. In certain embodiments, compositions comprising a tR1 peptide are useful for inhibiting a MARK family protein, inhibiting MARK2 function, inhibiting tau phosphorylation at Ser262, or in the treatment, prevention, or amelioration of neurodegenerative diseases, such as AD or frontotemporal dementia.

### Examples

In an effort to develop highly selective inhibitors of MARK2 function, alternative strategies for specifically targeting MARK2 proteins were explored. A peptide-based inhibitor was designed in the form of tR1, a peptide derived from the first repeat sequence of the hTau protein. While sequence variations on this peptide have been used extensively as a substrate for MARK2 in in vitro activation assays, this peptide has not been previously used as a selective inhibitor of MARK2 activity. These examples describe the development of tR1 as a direct inhibitor of MARK2, and show that this peptide can be used to block the MARK2-mediated phosphotylation of hTau in vitro and in cultured primary rat cortical neurons.

Rational Design of hTau R1 Domain Peptide Mimics

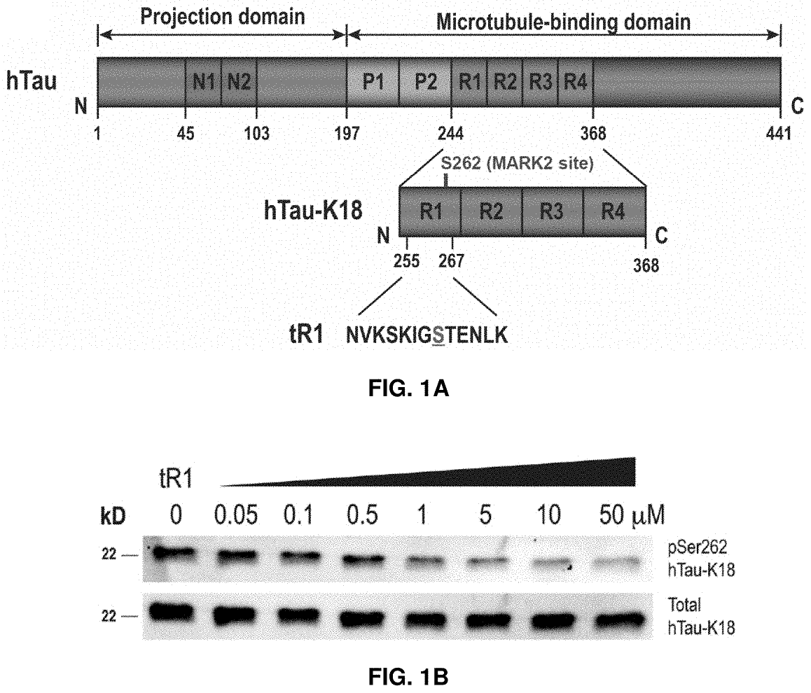

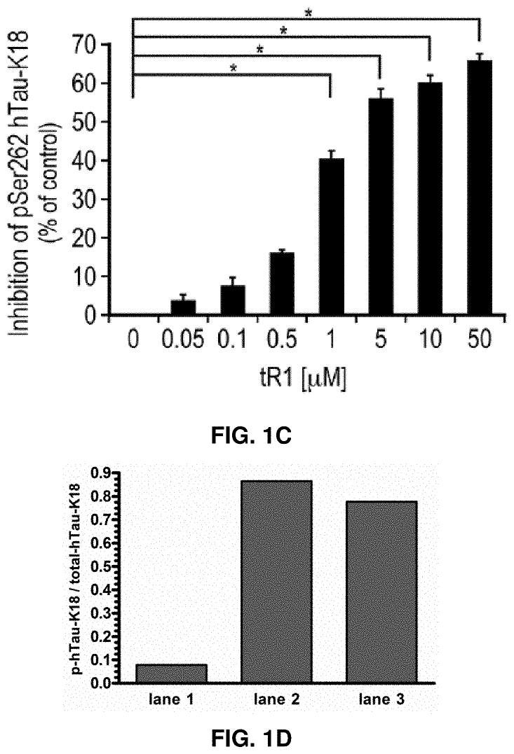

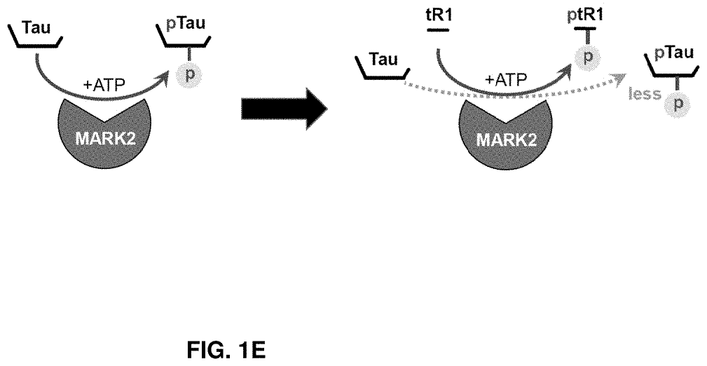

Efforts to develop a peptide-based inhibitor of MARK2 activity were initiated by designing a synthetic peptide mimic of the hTau repeat R1 domain. hTau is a 441-amino acid protein that contains two primary domains, an N-terminal projection domain, and a C-terminal microtubule-binding domain (FIG. 1A). The microtubule-binding domain contains four repeat domains (R1-4) that bind tubulin and act as substrates for MARK2. The R1 domain of hTau is a 33-residue sequence that contains the MARK2 recognition motif KIGS [SEQ ID NO: 3]; the Ser residue within this recognition motif corresponds to hTau Ser262 and is phosphorylated by MARK2. Similar peptides based on the R1 domain sequence have been used as substrates to validate the activity of MARK proteins in vitro. Alternatively, in the present example, the hTau R1 domain sequence was used as a model to develop a peptide-based inhibitor of MARK2 function. Analysis of the MARK2 crystal structure indicated that MARK2 binds the hTau R1 domain between residues 250 and 272 of the hTau sequence. Without wishing to be bound by theory, it is believed that a truncated version of the hTau R1 domain directly competes with hTau for the MARK2 active site and inhibits phosphorylation at Ser262. Therefore, a peptide that is a near-perfect sequence mimic of the hTau R1 sequence (FIG. 1A) was designed. This 13-residue peptide, designated tR1 (truncated R1), is shorter than the canonical R1 sequence by 20 residues, however it does contain a central KIGS [SEQ ID NO: 3] recognition motif flanked on each side by a short sequence of amino acids. These flanking residues are believed to enhance the binding affinity of tR1 to the R1 binding domain of MARK2, and increase the likelihood of successful kinase inhibition.

Characterization of Recombinant MARK2 and hTau-K18 Proteins

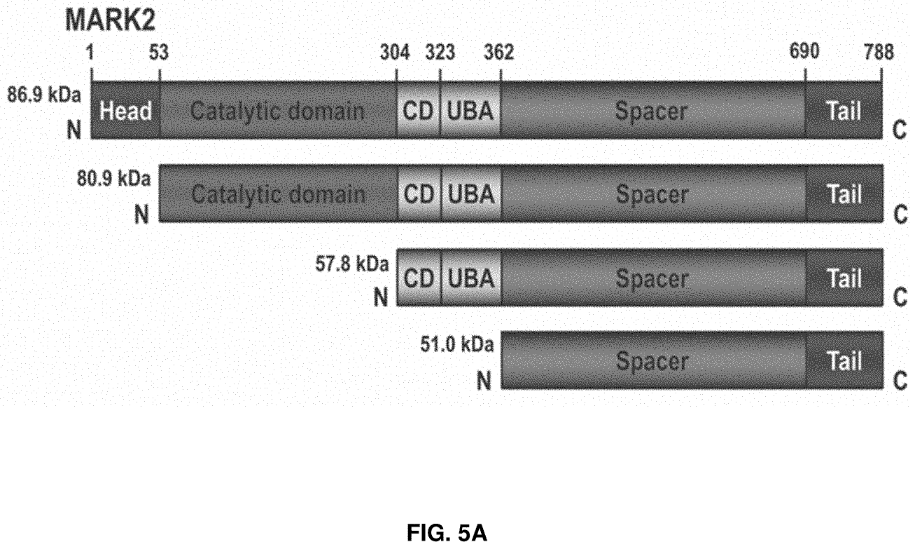

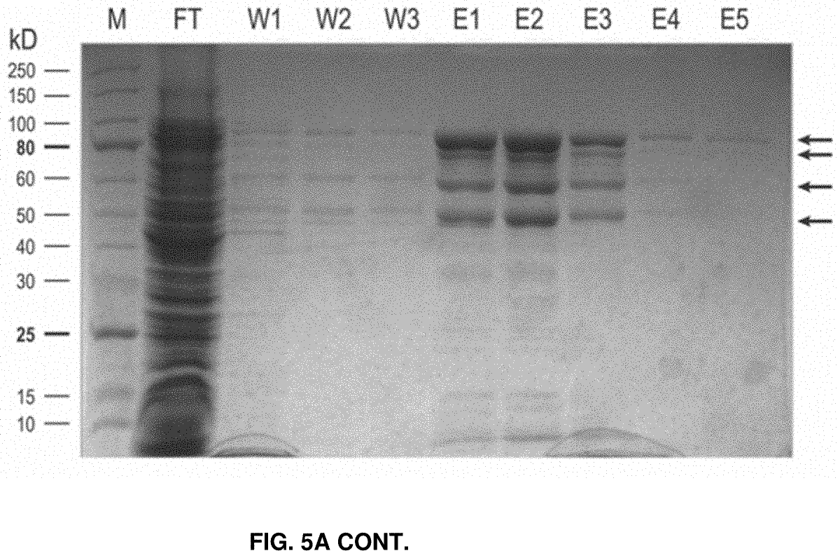

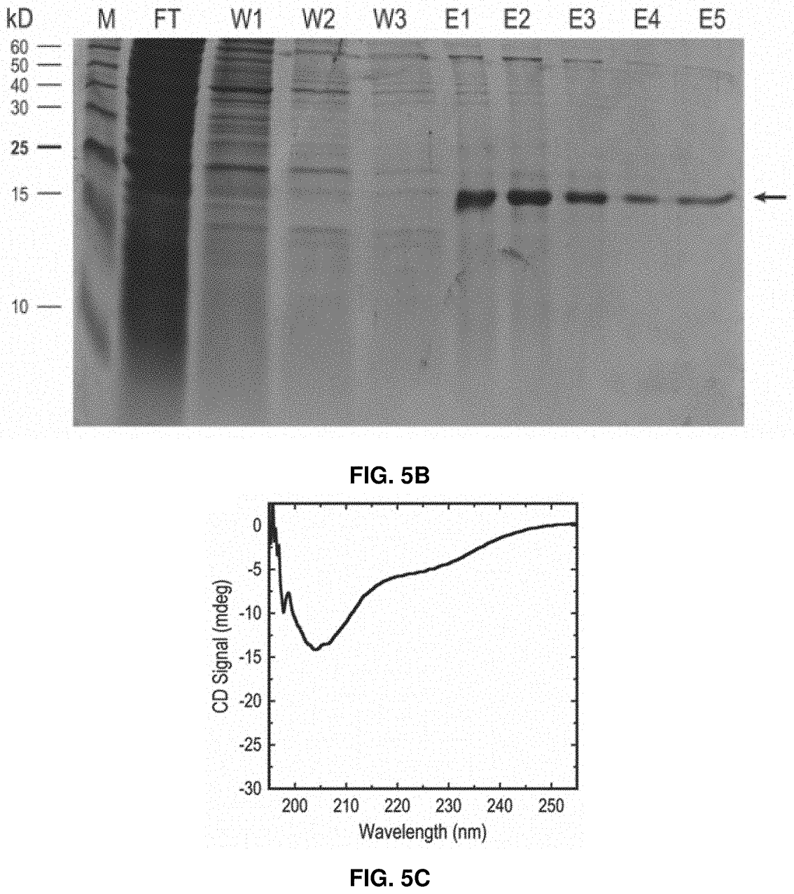

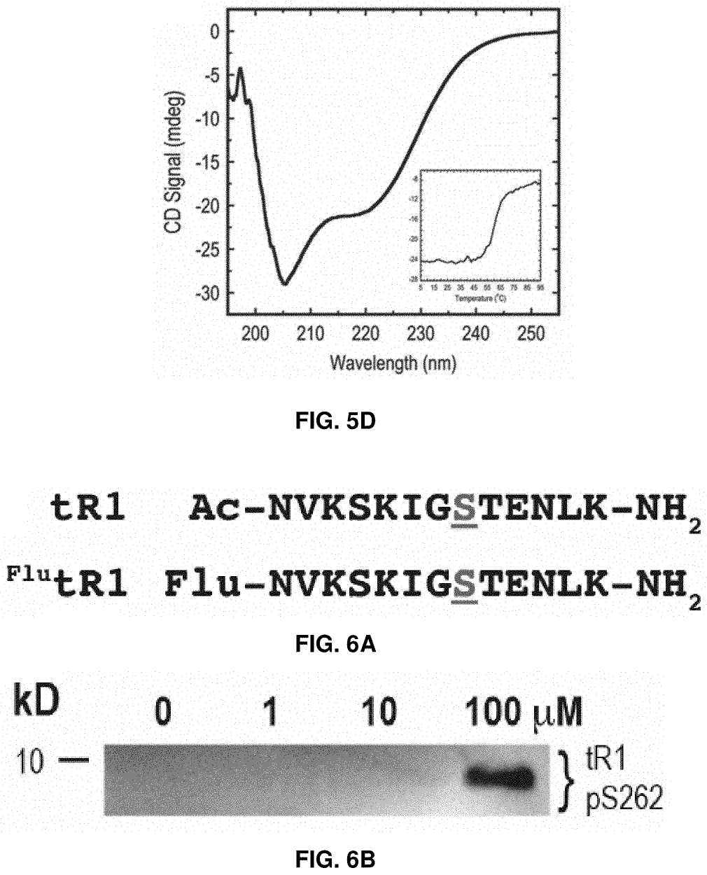

In vitro studies began by purifying recombinant MARK2 proteins from competent E. coli cells and evaluating its ability to phosphorylate hTau in vitro. Full-length MARK2 and a truncated isoform of hTau protein containing the four repeat domains (hTau-K18) expressed and purified from plasmids transformed into competent E. coli cells (FIGS. 5A-5B). All gene products were fully sequenced from their respective plasmids to ensure homology with reported MARK2 and hTau-K18 proteins. Following expression and purification, the proteins were dialyzed into phosphorylation buffer and the solution-phase structures of MARK2 and hTau were evaluated using wavelength-dependent circular dichroism (CD) spectropolarimetry. These experiments showed that MARK2 adopts a predominantly α-helical structure in solution with negative maxima at 206 and 222 nm, while hTau-K18 adopted a random coil structure in solution with a negative maximum at 201 nm and a slight shoulder at 225 nm (FIGS. 5C-5D). The thermal stability of MARK2 was determined using temperature-dependent CD and showed that the protein undergoes a cooperative unfolding transition at 61.1° C. (FIG. 5C, inset). hTau-K18 showed no cooperative transition upon thermal denaturation under similar conditions. Importantly, these results indicated that the recombinant MARK2 protein was well-folded and thermodynamically stable under conditions conducive to quantifying kinase activity.

In Vitro Characterization of M4RK2 Activity

The ability for MARK2 to phosphorylate hTau K18 was evaluated in vitro by incubating MARK2 with hTau-K18 in the presence or absence of ATP (FIG. 1B). All phosphorylation reactions were carried out for 60 minutes at 32° C. in phosphorylation buffer (50 mM Tris-HCl, 5 mM MgCl₂, 2 mM EGTA, 0.5 mM DTT, 0.5 mM benzamidine HCl, 0.5 mM PMSF, pH 8.0). Following the reaction, phosphorylated hTau-K18 proteins were separated by SDS-PAGE and identified by western blot using antibodies specific for hTau pSer262 (FIG. 1B, lanes 1 and 2). As a loading control, total hTau-K18 in each lane was quantified by western blot using antibodies specific for non-phosphorylated hTau-K18 (FIG. 1B, lower image). To ensure that the phosphorylation of hTau-K18 was a result of kinase activity and not auto-phosphorylation by ATP, hTau-K18 was incubated in the absence of MARK2 with or without ATP (FIG. 1B, lanes 3 and 4). These results showed no auto-phosphorylation of hTau-K18 in the absence of MARK2, further validating the efficacy of the recombinant MARK2 protein.

Time-course experiments indicated that MARK2 was capable of phosphorylating hTau-K18 after 30 minutes with maximal phosphorylation occurring after 60 minutes. Taken together, these results indicate that the recombinant MARK2 protein was active under these conditions and that hTau-K18 is a substrate for MARK2 activity. Additionally, it was shown that hTau-K18 is not phosphorylated by ATP alone, indicating that MARK2 is required to phosphorylate hTau-K18 at Ser262 in vitro.

Identification of tR1 as a Substrate for MARK2 In Vitro

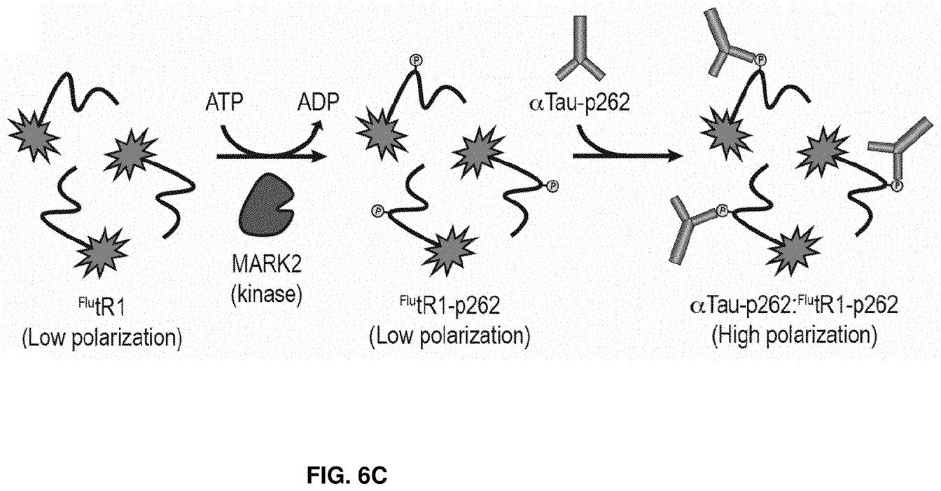

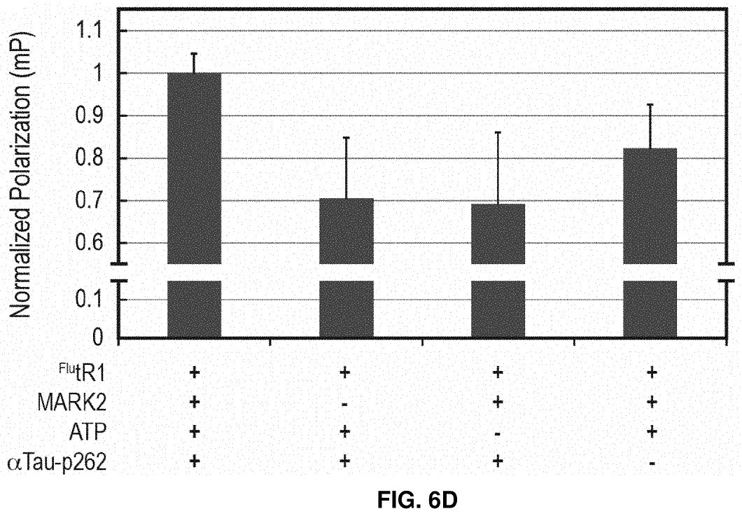

Once an otherwise competent kinase-substrate pair was developed for a working phosphorylation assay, tR1 was validated as a substrate for MARK2. The tR1 peptide (NVKSKIGSTENLK [SEQ ID NO: 1]) was synthesized using standard Fmoc-based solid-phase synthesis procedures and the final sequence was either acylated (tR1) or labeled with 5-carboxyfluorescein (tR1) on its N-terminus (FIG. 6A). See the experimental procedures, below, for details on the synthesis, labeling, purification and characterization of tR1 peptides. To evaluate whether tR1 is recognized as a substrate by MARK2, MARK2 was incubated with ATP in the presence of varying concentrations of tR1 peptide for 60 minutes at 32° C. in phosphorylation buffer. Following incubation, phosphorylated tR1 peptides were separated by SDS-PAGE and identified by Western blot using antibodies specific for hTau pSer262 (FIG. 6B). Western blot analysis confirmed tR1 as a substrate of MARK2 by the presence of a sharp band at the highest concentration (100 μM). Phosphorylation was not seen in reactions containing 0, 1, or 10 μM peptide, perhaps due to detection limits of the αTau-pSer262 antibody. Nevertheless, these experiments indicated that tR1 peptides can be phosphorylated by MARK2, and that the resultant epitope can be recognized by antibodies specific for hTau pSer262.

The ability for MARK2 to phosphorylate tR1 in vitro was further validated by incubating ^{Flu}tR1 peptides with MARK2 and ATP and monitoring the fluorescence polarization of the solution following addition of antibodies specific for hTau pSer262 (FIG. 6C). In these experiments, ^{Flu}tR1 peptides and MARK2 were incubated with and without ATP for 60 minutes at 32° C. in phosphorylation buffer. Following incubation, antibodies specific for hTau pSer262 were added to the reaction and allowed to incubate for an additional 60 minutes at room temperature. Due to the slower tumbling rate of an antibody-linked (and presumably phosphorylated) ^{Flu}tR1 peptide, it is believed that the fluorescence polarization of the phosphorylated tR1 peptide would be higher than that of its un-phosphorylated counterpart. Results from these experiments showed the polarization to be highest in solutions containing all reaction components, with lower polarizations measured in the absence of MARK2, ATP, or antibody (FIG. 6D), thus further validating tR1 as a substrate for MARK2 proteins.

Inhibiting MARK2 Activity with tR1 Peptides In Vitro

The ability for the tR1 peptide to act as an inhibitor of MARK2-mediated phosphorylation of hTau-K18 in vitro was examined. To test this, MARK2 and hTau-K18 were incubated in the presence of varying concentrations of tR1 for 60 minutes at 32° C. in phosphorylation buffer. Following the reaction, the proteins were separated by SDS-PAGE and identified by Western blot using antibodies specific for hTau pSer262 (FIG. 1C). Total hTau-K18 in each lane was quantified using antibodies specific for non-phosphorylated hTau-K18 (FIG. 1C, lower image). It can be seen from the results outlined in FIG. 1C that the tR1 peptide effectively inhibits MARK2-mediated phosphorylation of hTau-K18 at concentrations greater than 0.5 μM. Densitometry measurements indicate that treatment with 50 μM tR1 peptide resulted in a 66% inhibition of MARK2 activity (FIG. 1D). These results, coupled with the results showing that tR1 can act as a substrate for MARK2, strongly indicate that the tR1 peptide is capable of inhibiting MARK2 activity in vitro.

Evaluating tR1 Toxicity and Uptake Primary Rat Cortical Neurons

Given the in vitro results, the ability for tR1 peptides to inhibit MARK2-mediated tau phosphorylation in cultured cells was tested. Primary rat cortical neurons were chosen as a model system due to their robustness in culture and ability to express isoforms of MARK2 and tau that are homologous to their human counterparts. MARK2 expressed in rat conical neurons is over 90% homologous to the human MARK2, with 100% sequence similarity in the kinase domain. Furthermore, the 33-residue rat tau R1 domain is 97% homologous to the hTau R1 domain with only a single amino acid difference at position 257 (Lys257 in hTau, Arg190 in rat tau). Importantly, this minor sequence variation is not contained within the KIGS [SEQ ID NO: 3] recognition motif of the rat tau protein, and therefore should not impede inhibition of MARK2 by tR1 peptides.

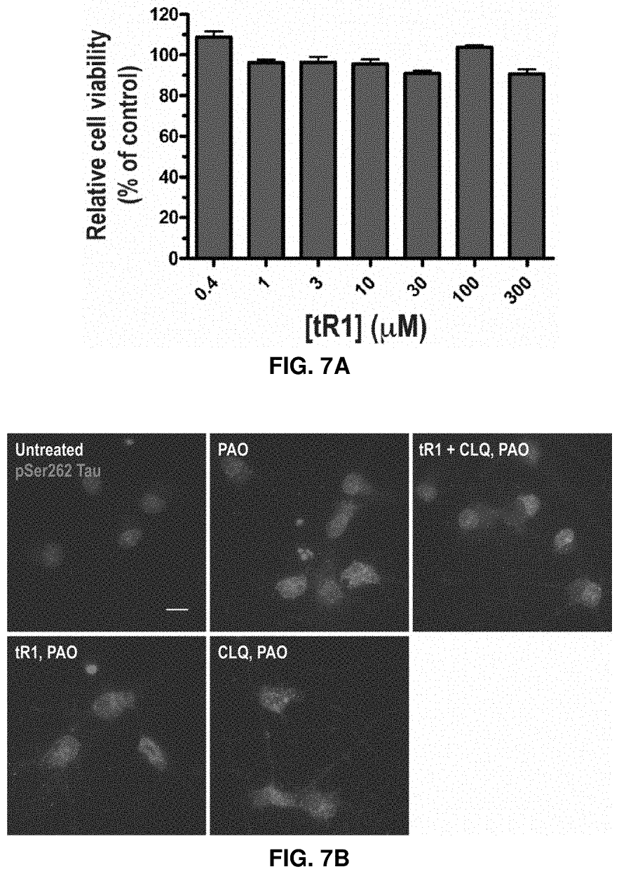

This series of experiments began by determining the cytotoxicity profile of tR1 in primary rat conical neurons. Primary cells were isolated from embryonic Sprauge-Dawley rats and cultured as described. Toxicity experiments were performed by treating the cells with varying concentrations of tR1 (0-300 μM) for 72 hours. Following incubation, cell viability was quantified using a colorimetric MTT assay (FIG. 7A). It was determined from this set of experiments that cell viability was not affected in treated cells compared to control (no treatment) cells, with only minimal (<5%) loss in cell viability at even the highest concentrations of tR1 peptide. Therefore, it was concluded that tR1 peptides are non-toxic to primary rat cortical neurons at concentrations up to 300 μM under these conditions.

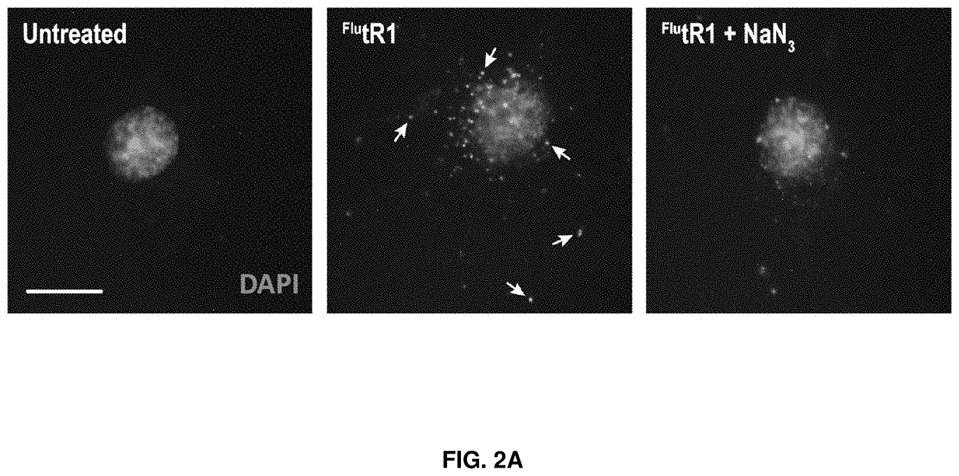

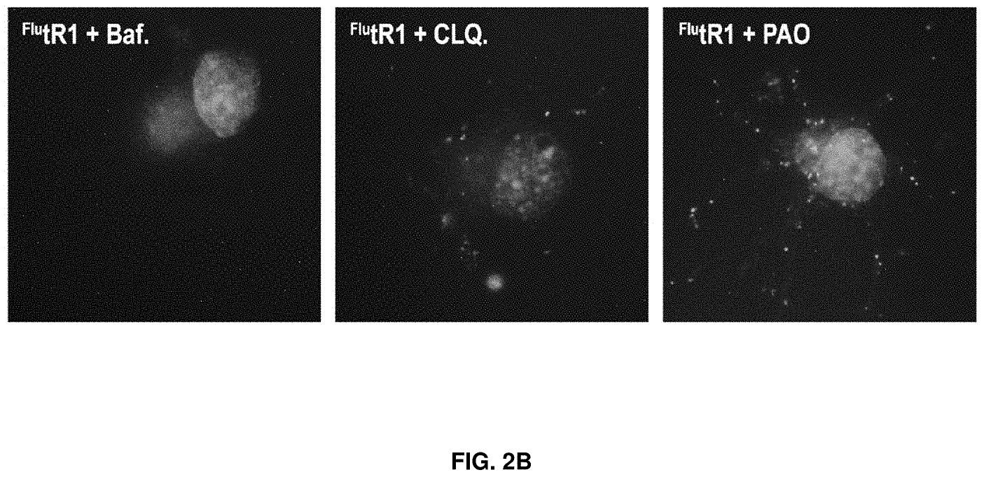

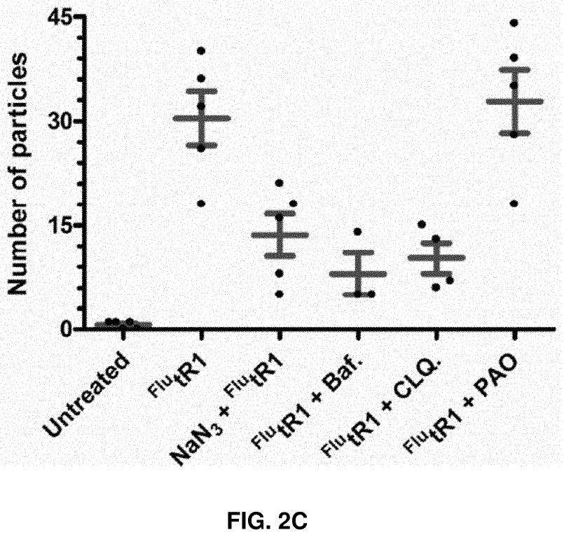

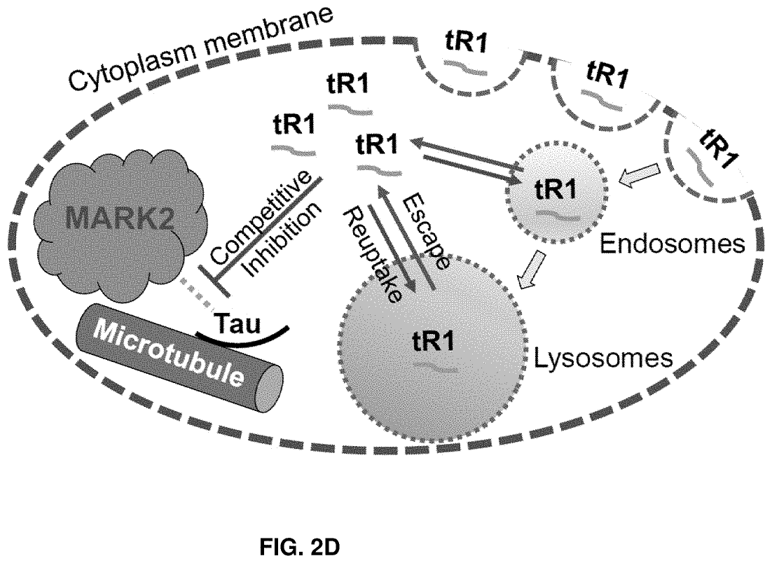

The tR1 peptide has a net charge of +2 at physiological pH and is expected to be cell-permeable under the treatment conditions. To evaluate the cell-permeability of tR1 peptides, primary rat conical neurons were treated with ^{Flu}tR1 and intracellular uptake was observed by fluorescence microscopy (FIGS. 2A-2B). For this series of experiments primary rat conical neurons were cultured for five to seven days to ensure maturation of neuronal processes. Mature neuronal cells were then incubated for 4 hours in media supplemented with 10 μM ^{Flu}tR1 peptide. Following incubation the cells were washed fixed and counter stained with DAPI to visualize the cell nuclei. It was determined from the uptake experiments that untreated cells showed no evidence of intracellular fluorescence under these conditions (FIG. 2A, left image). It was observed however that cells treated with 10 μM ^{Flu}tR1 peptides showed distinct fluorescent puncta within the cell body and neurites (FIG. 2A, middle image). Notably, cells co treated with 10 μM ^{Flu}tR1 and the metabolic inhibitor 10 mM sodium azide (NaN)showed a significant loss of fluorescent puncta within the cell body and neurites (FIG. 2A, right image). These data indicate that ^{Flu}tR1 are internalized through an ATP-dependent mechanism and do not passively diffuse into primary rat conical neurons.

To determine the mechanism of internalization we co treated cells with ^{Flu}tR1 and bafilomycin A1 (Baf), chloroquine (CLQ), or phenylarsine oxide (PAO) (FIG. 2B). Bafilomycins are a class of macrolide antibiotics that inhibit endosomal acidification in eukaryotic cells and have been used to facilitate the release of cell penetrating peptides from endosomal vesicles. Cells co treated with 10 μM ^{Flu}tR1 and 50 nM Baf showed no fluorescent puncta following treatment and diffuse green fluorescence within the cell soma (FIG. 2B, left image). CLQ is an aminoquinoline based therapeutic used in the treatment of malaria and causes endosomal rupture when administered to cells at high concentrations. Cells co-treated cells with 10 μM ^{Flu}tR1 and 100 μM CLQ (FIG. 2B, middle image) showed a significant loss of discrete fluorescent puncta and contained larger, more diffuse vesicular structures compared to cells treated with ^{Flu}tR1 alone (compare FIGS. 2A and 2B, middle images). PAO is an organometallic small molecule that is known to inhibit clathrin-mediated endocytosis. Interestingly, co-treating cells with 10 μM ^{Flu}tR1 and 5 μM PAO did not seem to affect the uptake of ^{Flu}tR1 (FIG. 2B, right image). In fact, there seemed to be little difference in the intracellular distribution of ^{Flu}tR1 in cells co-treated with ^{Flu}tR1 and PAO compared to cells treated with ^{Flu}tR1 alone (compare FIG. 2A middle image and FIG. 2B right image). Taken together, these data indicate that ^{Flu}tR1 peptides are permeable in rat primary conical neurons and are internalized via a clathrin-independent mechanism. This conclusion is supported by the observation that cells co-treated with ^{Flu}tR1 and the endosome acidification inhibitors Baf and CLQ display diffuse patterns of fluorescence throughout the cell body compared to cells treated with ^{Flu}tR1 alone. In addition, PAO had no effect on cell uptake, indicating that ^{Flu}tR1 can be internalized through some form of clathrin-independent mechanism. Finally, these findings show that ^{Flu}tR1 peptides are distributed throughout the cell soma and neurites once inside the cell, which may facilitate the delivery of ^{Flu}tR1 to regions of the cell interior containing MARK2.

Inhibiting Tau Phosphorylation in Primary Rat Cortical Neurons with tR1 Peptides

Once it was established that tR1 is minimally cytotoxic and cell-permeable, the ability for tR1 peptides to inhibit MARK2-mediated phosphorylation of endogenous tau proteins in primary rat cortical neurons was evaluated. It has been demonstrated that treatment of cells with PAO, an organometallic compound that reacts with vicinal thiols, increases the selective phosphorylation of tau at Ser262 and Ser356 within its R1 and R4 microtubule binding domains. Studies have demonstrated that PAO facilitates MARK2-mediated phosphorylation of tau at these specific residues. While the exact mechanism of PAO-mediated MARK2 activation has yet to be elucidated, it is thought that PAO stimulates signaling pathways that result in phosphorylation of MARK at select Ser and Thr residues crucial for kinase activity.

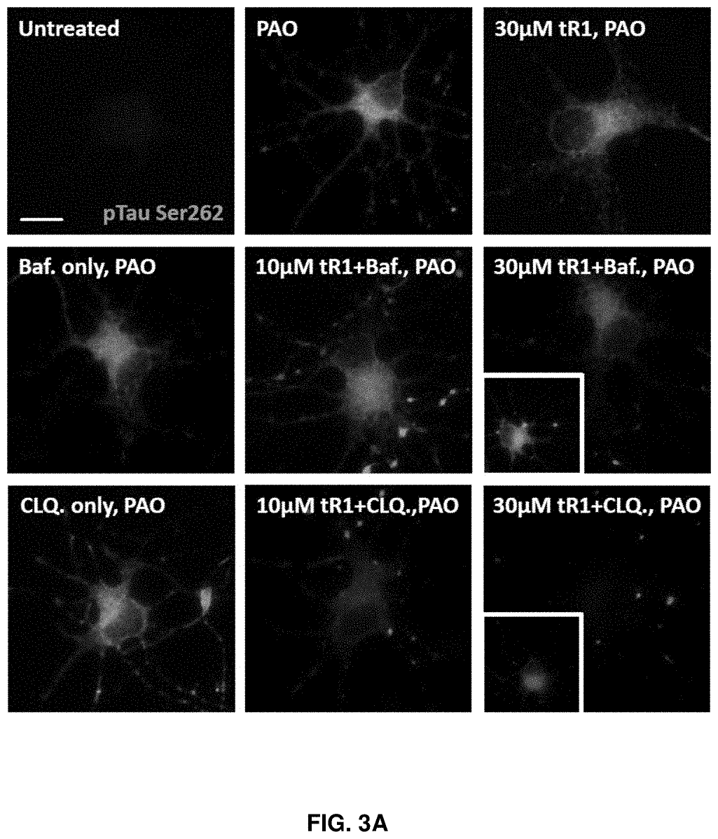

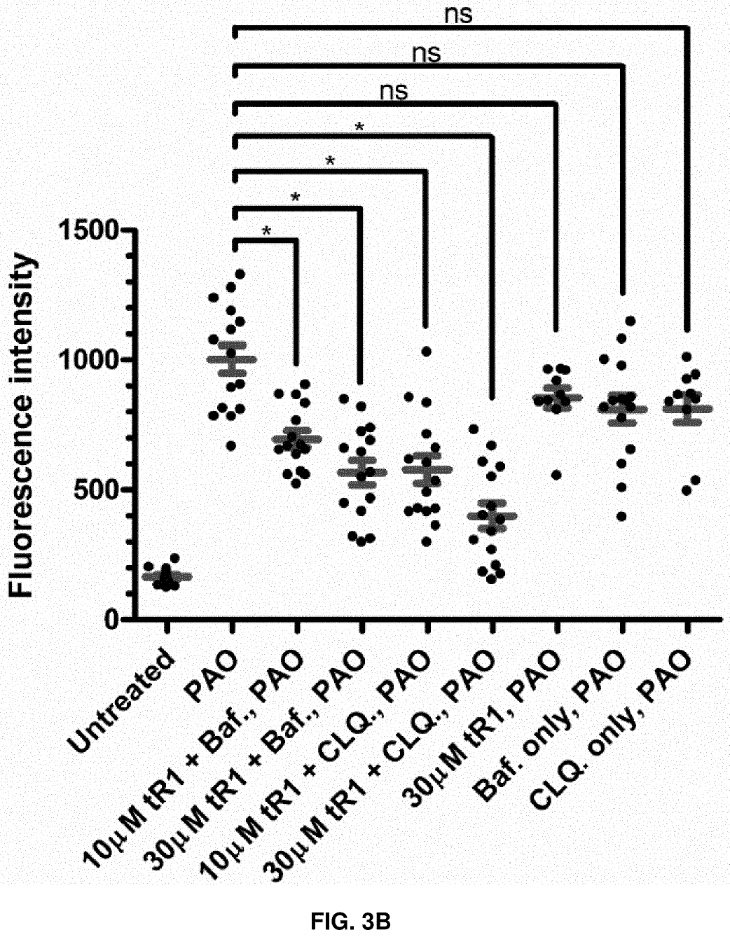

Inhibition studies were performed on cultured primary rat cortical neurons that were grown in media for five to seven days to ensure maturation of cellular processes. MARK2-mediated phosphorylation of tau was initiated by treating cells is ith 5 μM PAO and the extent of phosphorylation was quantified using fluorescently-labeled antibodies against pTau Ser262 (FIG. 3). Surprisingly, it was observed that treatment of the cells with tR1 peptides alone did not inhibit MARK2-mediated phosphorylation of tau under these conditions (FIG. 3A, top row). It has been shown that ^{Flu}tR1 is taken up in primary rat cortical neurons (FIG. 2), therefore, it is believed that the tR1 peptides are not effective in inhibiting MARK2-mediated phosphorylation of endogenous tau because they became trapped in endosomes and are unable to reach their cytosolic target. To facilitate endosomal release of tR1 peptides, cells were co-treated with varying concentrations of tR1 and 50 μM bafilomycin A1 (FIG. 3A middle row) or 100 μM chloroquine (FIG. 3A bottom row). Neither bafilomycin A1 nor chloroquine treatments alone affected PAO-induced MARK2 mediated phosphorylation of tau. However it is seen that co-treatment of tR1 peptide with either bafilomycin A1 or chloroquine significantly reduced the extent of tau phosphorylation in rat primary cortical neurons (FIGS. 3A and 3B). The observed inhibitory effects of tR1 were concentration dependent, with 10 μM tR1 resulting in modest reduction in tau phosphorylation and 30 μM tR1 showing near complete inhibition of MARK2-mediated phosphorylation of tau proteins. Interestingly, inhibition was greatest in cells co-treated with tR1 peptides and chloroquine, indicating that endosomal rupture may facilitate delivery of the tR1 peptide to the cytoplasm where it can exert its inhibitory effects.

The ability for tR1 to inhibit MARK2-mediated phosphorylation of tau was also directly evaluated in rat primary cortical neurons using western blot.

The tR1 Peptide does not Inhibit Phosphorylation of Tau at Residue Thr231

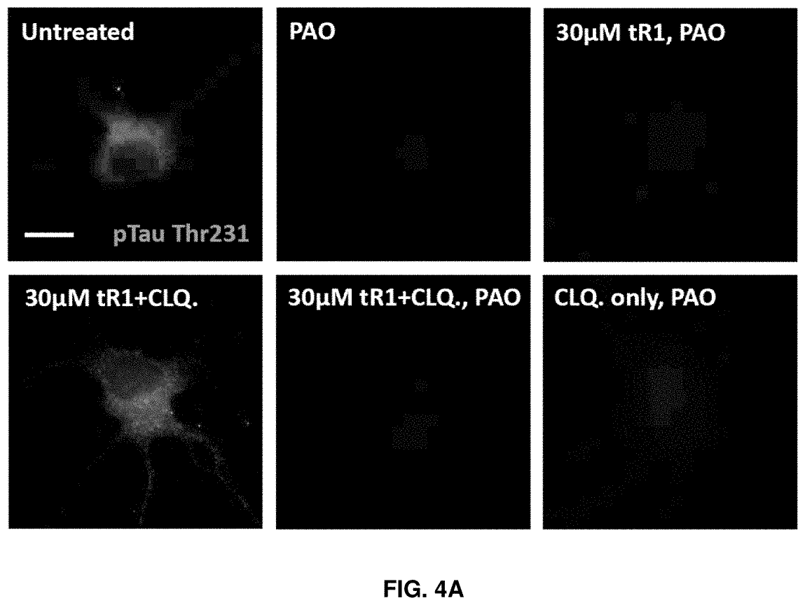

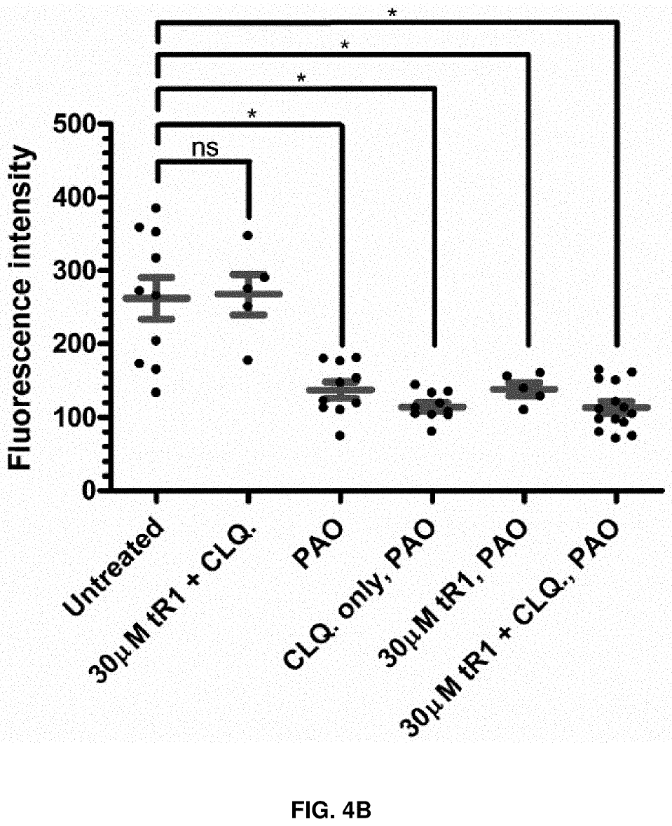

In order to gain insight into the selectivity of the tR1 peptide, the ability for tR1 peptide to inhibit the activity of kinases that phosphorylate tau at sites other than Ser262 in rat primary cortical neurons was evaluated. To accomplish this, the ability for tR1 peptides to inhibit kinases that phosphorylate tau at Thr231 was tested (FIGS. 4A-4B). There are several known kinases that phosphorylate tau at Thr231 including GSK-3β, CDK2, and CDK5. To test the effects of tR1 on the activity of these kinases, cultured rat primary cortical neurons were co-treated with 30 μM tR1 and 100 μM CLQ, and the level of pTau Thr231 was quantified by immunofluorescence (FIG. 4A). CLQ was included in these treatments to ensure delivery of tR1 to the cytoplasm. The results showed that cells treated with tR1 and CLQ did not show any significant loss of pTau Thr231 compared to untreated control cells. These data indicate that tR1 peptides do not inhibit protein kinases that phosphorylate tau at Thr231.

Following confirmation that tR1 peptides do not directly inhibit kinases that phosphorylate tau at Ser231, the affects that tR1 peptides had on the deactivation of kinases by PAO in rat primary cortical neurons were evaluated. To test this, cultured rat primary cortical neurons were treated with varying combination of PAO, tR1 and CLQ, and phospho-tau Thr231 levels were observed by immunofluorescence (FIG. 4A, right image). It was observed in these experiments that PAO treatment alone completely abolished phosphorylation at tau Thr231. This result indicates that PAO either inactivates kinases that phosphorylate this position of tau Thr231 or activates phosphatases that remove this specific phosphorylation. Perhaps more importantly, however, it was observed that tR1 did not influence the PAO-mediated inhibition of tau Thr231 phosphorylation even in the presence of CLQ (FIG. 4A-4B). These results indicate that tR1 did not simply interact with PAO, as tR1 failed to prevent PAO-mediated inhibition. Furthermore, these results indicate that PAO-induced kinase inhibition was not mediated through MARK2 activation, as inhibiting MARK2 with tR1 did not affect levels of pTau Thr231.

In Vitro Peptide Stability Assays

Stock peptide solutions for use in stability assays were prepared by dissolving tR1 to a final concentration of 5 mg/mL in dimethyl sulfoxide (DMSO). For stability tests, 2 μL of stock tR1 peptides were added to 198 μL pre-warmed RPMI medium supplemented with 25% (v/v) heat-inactivated human AB serum or neurobasal (NB) media at a final concentration of 50 μg/mL. Each reaction was subsequently incubated at 37° C. for the indicated times. Following incubation, 400 μL aqueous trichloroacetic acid (15%, w/v) was added to each reaction and the samples were immediately cooled on ice for 15 minutes. The samples were then centrifuged 3× at 14,000 rpm to remove precipitated proteins. As a positive control for enzymatic degradation, the tR1 peptide was mixed with trypsin (10 ng/μL) at a final concentration of 1.0 μg/μL in 100 μL digestion buffer (100 mM Tris, 1 mM CaCl₂, pH 7.8) and was allowed to incubate at 37° C. for 15 minutes. Following the reaction, 100 μL of aqueous TFA (50%, v/v) was added to stop proteolysis. For analysis, the reaction mixtures for each assay were loaded onto a reversed-phase analytical C18 column (Grace, 5 μm, 50×2.1 mm) and eluted within 20 minutes using a linear gradient of 0-50% solvent B (0.1% TFA in ACN) over solvent A (0.1% TFA in water). The background signal was subtracted from each respective spectrum and the fraction of intact peptide was quantified from product peak integration normalized to undigested controls. HPLC data were acquired using OpenLab CDS ChemStation Software v1.06 (Agilent) and processed using KaleidaGraph v4.5 (Synergy Software).

The tR1 Peptide is Stable in RPMI and Neurobasal Media

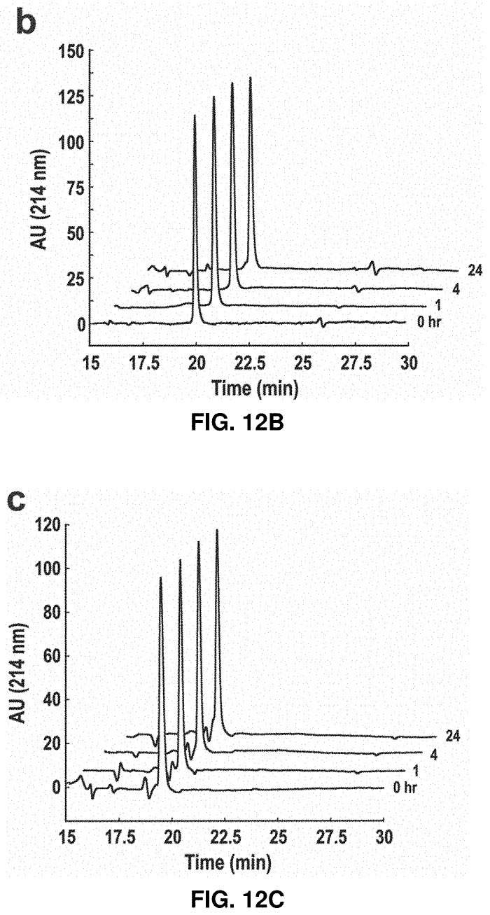

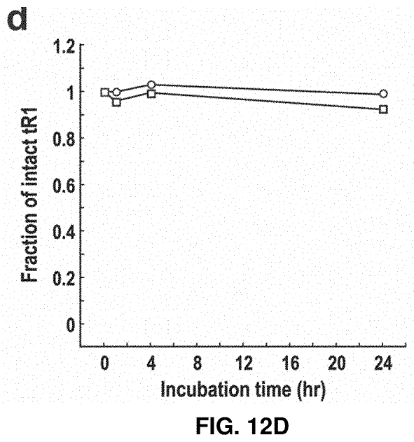

A major limitation to the therapeutic efficacy of peptides is their susceptibility to proteolytic degradation. Indeed, short peptides generally have a serum half-life measured in minutes. The in vitro stability of tR1 peptides in the presence of proteolytic enzymes was determined. To evaluate peptide stability, we incubated tR1 in RPMI media supplemented with 25% (v/v) human serum or in NB medium for up to 24 hours at 37° C. (FIGS. 12A-12D). As a positive control for degradation, tR1 peptides were incubated with trypsin (10 ng/μL) in digestion buffer (FIG. 12A). Following the incubation, the reactions were quenched using haloacetic acids and the extent of degradation was quantified by analytical HPLC. The tR1 peptide was rapidly degraded by trypsin, which showed complete degradation of the peptide after 15 minutes of incubation (FIG. 12B). Surprisingly, it was found that the tR1 peptide did not degrade in either RPMI media supplemented with human serum or NB media at treatment times up to 24 hours (FIG. 12C). Results here show that tR1 remains >90% stable in RPMI medium supplemented with human serum and NB media under these conditions (FIG. 12D).

Discussion

The ability to selectively inhibit protein kinases represents one of the most significant challenges in modern drug development. Classical protein kinases, despite sharing a common ‘catalytic fold’ that binds ATP, display remarkable diversity when binding to protein substrates. This diversity is primarily attributed to sequence and structural variations in regulatory domains located outside the catalytic core that directly affect the interaction of the kinase with its protein substrate. Protein kinase activities are often controlled through protein-protein interactions that can occur between large globular domains or through peptide epitopes that bind docking grooves within the protein kinase. Peptide-based molecules that mimic discrete epitopes of protein substrates are emerging as powerful tools for the targeted inhibition of kinase activity.

MARK2 proteins function by phosphorylating residues within the repeat domains of tau, which, in turn, directly influences the ability of tau to associate with microtubules. This example describes the development of a peptide-based inhibitor of MARK2 protein function, tR1. The hyper-phosphorylation of human tau by MARK family kinases can lead to various neurodegenerative disorders including AD and frontotemporal dementia. The ability to selectively inhibit MARK protein kinase function represents a major therapeutic challenge for researchers and clinicians. MARK2 proteins phosphorylate tau at numerous sites, however only a select few directly impact tau association with microtubules. The phosphorylation profile of the KXGS [SEQ ID NO: 2] motifs within the tau repeat domains plays a crucial role in modulating tau interactions with microtubules. The MARK2 phosphorylated Ser262 within the tau R1 domain has been implicated in controlling the physiological function of tau protein. Despite the immense physiological importance of MARK2, only a small number of inhibitors of this kinase exist. In fact, to the best of our knowledge, 39621 remains the only commercially available small molecule inhibitor of MARK activity and is not selective for discrete proteins within the MARK family. To develop highly selective inhibitors of MARK2 function and expand the repertoire of MARK inhibitors beyond small molecule ligands, a peptide-based inhibitor of MARK2 that functions to inhibit MARK2 activity in vitro and in live cells was developed.

The tR1 molecule was developed by mimicking the tau protein R1 domain, and represents a novel class of peptide-based MARK2 inhibitor. This peptide was developed by mimicking the tau R1 domain sequence that is known to interact with the MARK2 active site. The tR1 peptide includes the KIGS [SEQ ID NO:3] recognition motif and contains a Ser residue that corresponds to Ser262 within the full-length tau protein known to be phosphorylated by MARK2. In addition, the tR1 sequence includes 4-5 amino acids on either side of the canonical Ser262 residue that likely enhance the specificity of this peptide to MARK2. The inhibitory effect of tR1 was evaluated in vitro using standard phosphorylation assays that included MARK2 and a truncated version of the human tau protein hTau-K18. These results showed that tR1 peptides can inhibit MARK2 activity up to 66% at a concentration of 50 μM. It was also determined through in vitro and fluorescence polarization studies that tR1 acts as substrate for MARK2, becoming phosphorylated in the presence of MARK2 and ATP. Collectively, these results indicate that tR1 acts as a direct inhibitor of MARK2 function in vitro, competing with the tau R1 domain sequences for the MARK2 active site.

Subsequent studies in live cells revealed that the tR1 peptide is non toxic to rat primary cortical neurons at concentrations up to 300 μM, a concentration well above the established in vitro inhibitory concentration of 10 μM. It was also demonstrated that fluorescently-labeled tR1 peptides are cell permeable in primary neurons following a 4 hour incubation, with the peptides concentrating in distinct puncta throughout the cell soma and neurite outgrowths. Pre-treating cells with the metabolic inhibitor NaN₃ significantly reduced uptake of tR1 peptides compared to cells treated with ^{Flu}tR1 alone, further validating the conclusion that tR1 is internalized via endocytosis and not passive diffusion in primary rat cortical neurons. Furthermore, co-treatment of cells with ^{Flu}tR1 and endosome acidification inhibitors bafilomycin or chloroquine resulted in the abolishment of distinct fluorescent puncta within the neurons. Cells co-treated in this manner showed a more diffuse pattern of green fluorescence throughout the cell soma and larger fluorescent vesicles than their untreated counterparts, indicating that the ^{Flu}tR1 peptides were delivered to the cytosol upon co-treatment with endosome disruptors. Interestingly, pre-treating cells with phenylarsine oxide, which is known to inhibit clathrin-mediated endocytosis, did not affect internalization of ^{Flu}tR1 peptides. Collectively, these data reveal that ^{Flu}tR1 peptides are internalized by rat primary cortical neurons through clathrin-independent mechanism and localize in cell soma upon release from endosomes.

The tR1 peptide was also shown to inhibit PAO-mediated activation of MARK2 by immunofluorescence assays. Previous reports have shown that PAO is able to stimulate pathways that result in the activation of MARK2, and subsequent phosphorylation of tau at Ser262 and Ser356. Treatment of rat-primary neurons with tR1 resulted in a significant reduction of tau pSer262 in cells treated with PAO. This inhibition was concentration-dependent, with 30 μM tR1 exerting a more pronounced effect than 10 μM tR1. It should also be noted that the tR1 peptide exerted its inhibitory effects only when co-treated with the endosome disruptors bafilomycin or chloroquine. Interestingly, cells co-treated with 30 μM peptide and bafilomycin resulted in approximately a 50% loss in signal compared to cells treated with PAO alone, while cells co-treated with 30 μM peptide and chloroquine showed a loss in signal comparable to that of baseline (untreated cells). These results indicate that tR1 peptides are capable of inhibiting MARK2-mediated phosphorylation of endogenous tau Ser262 in primary rat cortical neurons when released from endosomes. The phenomenon of endosomal entrapment is a common issue with many cell-penetrating peptides, and tR1 variants appended with amino acid sequences known to facilitate endosomal escape can be developed. The ability for tR1 to inhibit MARK2 activity in rat cortical neurons was also evaluated using western blot techniques. See FIG. 4C.

Finally, the results showed that tR1 does not inhibit other kinases that phosphorylate tau at Thr 231. GSK-3β is known to phosphorylate tau protein at Thr231, which plays a critical role in regulating tau's ability to bind and stabilize microtubules. PAO is a putative inhibitor of tyrosine phosphatase activity, and has been shown to inactivate GSK-3β in mouse neuronal cells. In the system in this example, PAO serves divergent roles activating MARK2 and deactivating GSK-3β. It is shown in this example that cells treated with PAO alone contained significantly less pThr231 tau compared to untreated cells. Co-treatment of the cells with tR1 and PAO also showed a significant loss in pThr231 levels compared to untreated controls. Furthermore, similar results were observed in cells treated with PAO, tR1, and the endosome disrupters bafilomycin and chloroquine, indicating that cytosolic tR1 had no impact on the level of phosphorylated Thr321 in PAO treated rat primary conical neurons. Collectively, these results indicate that tR1 does not inhibit PAO from inactivating GSK-3β and PAO-induced GSK-3β inhibition was not mediated via MARK2 activation, as inhibiting MARK2 with tR1 had no effect on the phosphorylation levels of Thr231.

The results from this example have revealed that MARK2 protein activity can be inhibited by a peptide-based sequence mimetic of the tau R1 domain. Thus, MARK2-mediated activity has been suppressed by a peptide (and not small molecule) inhibitor. On a broader scale, this example serves to expand the repertoire of molecules that can be used to inhibit MARK2-mediated tau phosphorylation and represents next-generation therapeutics designed to treat neurodegenerative disorders such as Alzheimer's disease and frontotemporal dementia. tR1 molecules and their derivatives, including peptide-based mimetics of other R domains, may also be used as tools to help better understand the complex nature of MARK2 biology and the specific role of discrete phosphorylation sites within the repeat domains of tau.

Also shown are that tR1 peptides are highly stable in RPMI media supplemented with human serum and in NB medium. It was demonstrated that tR1 peptides were >90% intact after incubation in each respective media for 24 hours. On the contrary, the tR1 peptide in buffer containing trypsin was rapidly degraded in less than 15 minutes, which is believed to be due to the number of lysine residues contained within the tR1 sequence. Importantly, the remarkable temporal stability in NB media increases the likelihood that tR1 peptides remain intact as they are delivered to cultured neurons. These stability assays were conducted in media only and therefore do not reveal how long the tR1 peptide survives in an intracellular environment. These assays do show that tR1 is stable in culture media for times appropriate for efficient uptake and cytosolic delivery.

Experimental Procedures

Reagents and Chemicals

All Fmoc-protected amino acids, Fmoc-PAL-AM resin, and coupling reagents were obtained from Novabiochem (Billerica, Mass.). N,N-diisopropylethylamine (DIEA), N-methyl-2-pyrrolidone (NMP), piperidine, isopropyl-D-l-thiogalactopyranoside (IPTG), ammonium persulfate, sodium azide, Tween-20, benzamidine HCl, sodium phosphate (monobasic) monohydrate, 3-[(3-cholamidopropyl)-dimethylammonio]-1-propanesulfonate hydrate (CHAPS), bovine serum albumin, adenosine-5′-phosphate disodium salt hydrate (ATP), 2-mercaptoethanol, triisopropylsilaine (TIPS), N-methylmorpholine (NMM), bafilomycin A1, chloroquine, sodium azide, phenylarsine oxide, acetic anhydride, trichloroacetic acid, Triton X-100, phenylmethanesulfonyl fluoride (PMSF), ethylene glycol-bis (2-aminoethylether)-N,N,N′,N′-tetraacetic acid (EGTA), trypsin and rabbit anti-(tau) (#T6402) were purchased from Sigma-Aldrich (St. Louis, Mo.). Glycine and chloramphenicol were obtained from Calbiochem® EMD Millipore Corp (Billerica, Mass.), n-Butanol, LB Medium, and coomassie brilliant blue R-250 were obtained from MP Biomedicals (Santa Ana, Calif.). Tris (base), bisacrylamide, sodium dodecyl sulfate, tetramethylethylenediamine (TEMED), methanol, phenol, imidazole, LB agar, sodium chloride, ampicillin, RPMI Medium 1640 bacterial protein extraction reagent (B-PER), and protease inhibitors were purchased from Thermo Fisher Scientific (Waltham. Mass.). Human AB serum (35-060-CI) was purchased from Corning Inc. (Corning, N.Y.). Potassium chloride, potassium phosphate, calcium chloride, 2-propanol, and magnesium chloride hexahydrate were obtained from Fisher Scientific (Fair Lawn, N.J.). Neurobasal (NB) medium and B27 supplement were purchased from Invitrogen (Carlsbad, Calif.). L-glutamine and amphotericin B were purchased from Calbiochem (Darmstadt, Germany). Protein marker was obtained from New England Biolabs (Ipswitch, Mass.). Trifluoroacetic acid (TFA) was purchased from Acros Organics (Morris Plains, N.J.). 5-carboxyfluorescein (5-CF) and dimethyl sulfoxide (DMSO) were obtained from Santa Cruz Biotechnology (Dallas, Tex.). Acetonitrile (ACN) was purchased from NeoBits (Sunnyvale, Calif.). Ni-NTA agarose resin was purchased from Molecular Cloning Laboratories (San Francisco, Calif.). Bromophenol blue was obtained from Eastman (Kingsport, Tenn.). Amersham ECL Prime Western Blotting Detection Reagents were obtained from GE Health Care (Pittsburgh, Pa.). Precision Plus Protein™ Standards Dual Color, immun-Blot® PVDF Membrane were purchased from (Hercules, Calif.) Bio-Rad. Coomassie brilliant blue G-250 was purchased from Bio-Rad (United Kingdom). Rabbit polyclonal anti-Tau (Phospho 5262) antibody (#ab4856) was obtained from Abcam. Goat anti-rabbit IgG (HRP) was purchased from Abcam (Cambridge, Mass.). All other chemical reagents were obtained from commercial sources and used without any further modification unless stated otherwise.

Peptide Synthesis and Purification

Peptides were synthesized using standard solid phase peptide synthesis protocols on Fmoc-PAL-AM resin (25 μmol scale). All amino acid couplings and deprotections were performed in a microwave accelerated reaction system (CEM) using a fritted glass reaction vessel to facilitate washing of the resin. Multiple washing steps using fresh NMP were performed in between each coupling and deprotection reaction described below. Deprotection of terminal amino acids was achieved by treating the resin-bound peptide with 25% piperidine (v/v) in NMP containing 0.1M HOBt to minimize aspartimide formation. Following deprotection and washing, coupling reactions were performed by the adding 5 equivalents (eq) amino acid, 5 eq PyClock and 10 eq DIEA in NMP to the resin. All eq values are based on the resin loading level. Iterative coupling and deprotection cycles were performed until peptides of desired sequence were achieved. Following completion of the synthesis, resin-bound peptides were either acylated or labeled on their N-terminus with 5-carboxyfluorescein (5-CF). Acylations were performed by reacting Fmoc-deprotected peptides in a solution of 6% (v/v) acetic anhydride and 6% (v/v) NMM in NMP. This reaction was allowed to stir for 15 minutes at room temperature and repeated before final washing steps otlined below. Fluorescent-labeling was performed by preparing a solution of 3 eq 5-CF, 3 eq HCTU and 7.5 eq DIEA in NMP and adding it to the resin bound peptide. The labeling reaction was allowed to stir in the dark at room temperature for 24 hours. Following acylation or labeling with 5-CF, the resin bound peptides were washed thoroughly with NMP and DCM and dried under vacuum to remove residual solvent. Following drying, resin bound peptides were then globally deprotected and cleaved from the resin by adding a solution composed of TFA/water/phenol/TIPS (88/5/5/2, v/v/v/v) to the resin. This reaction was allowed to proceed for 30 min at 38° C. Once the reaction was complete, the peptide was precipitated in cold diethyl ether, pelleted and resuspended in a suitable volume of 15% ACN in water. This solution was then frozen and the peptides were lyophilized to dryness.

For purification, crude peptide powders were resuspended in a suitable volume of 15% (v/v) ACN in water. Once dissolved, the peptides were purified by HPLC across a reversed-phase preparative-scale C18 column (Grace, 10 μM, 250×10 mm). Peptides were separated using a linear gradient of 15-50% solvent B (0.1% TFA in ACN) over solvent A (0.1% TFA in water) at a flow rate 4 mL/min. All major peak fractions were collected and analyzed by mass spectrometry to confirm the peptide identity. Fractions containing the purified peptide products were combined, frozen and lypohilized twice. For storage, purified peptide powders were re-dissolved in 100% water and stored at 4° C. protected from light. The concentrations of the pure peptide were determined by dry weight (acylated peptide) or using an extinction coefficient of 38,000 M⁻¹cm⁻¹ at 450 nm in water (fluorescently-labeled peptide).

Characterization of Peptides by Analytical HPCL and Mass Spectrometry

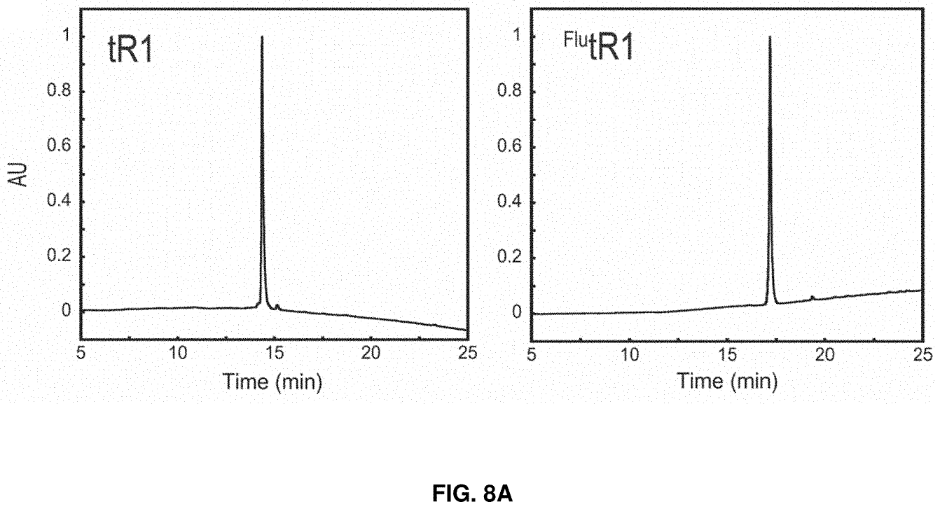

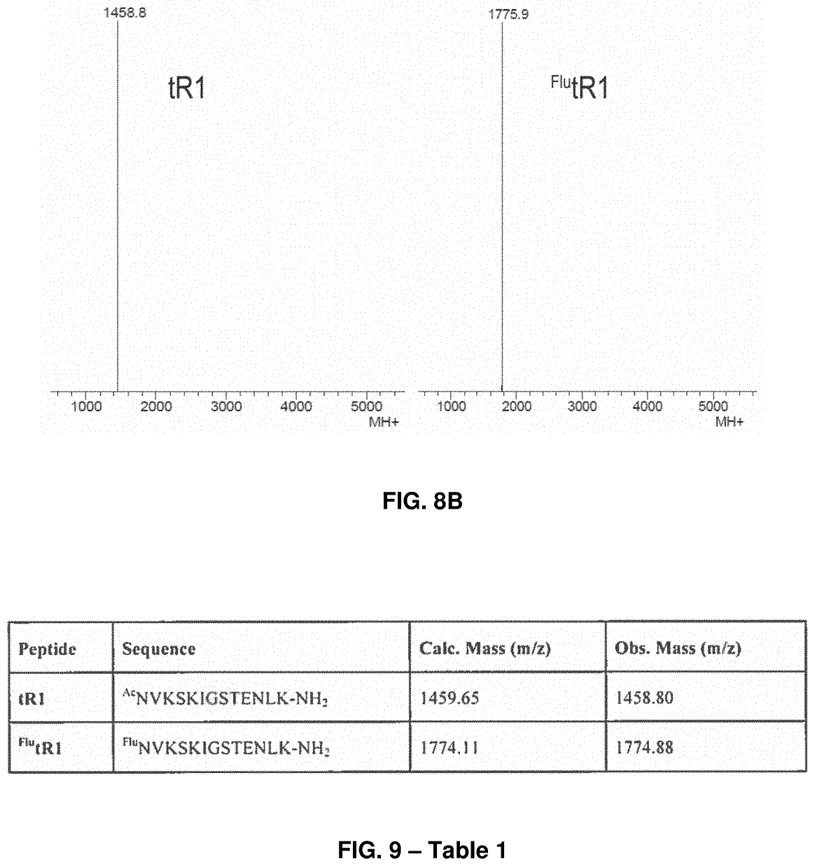

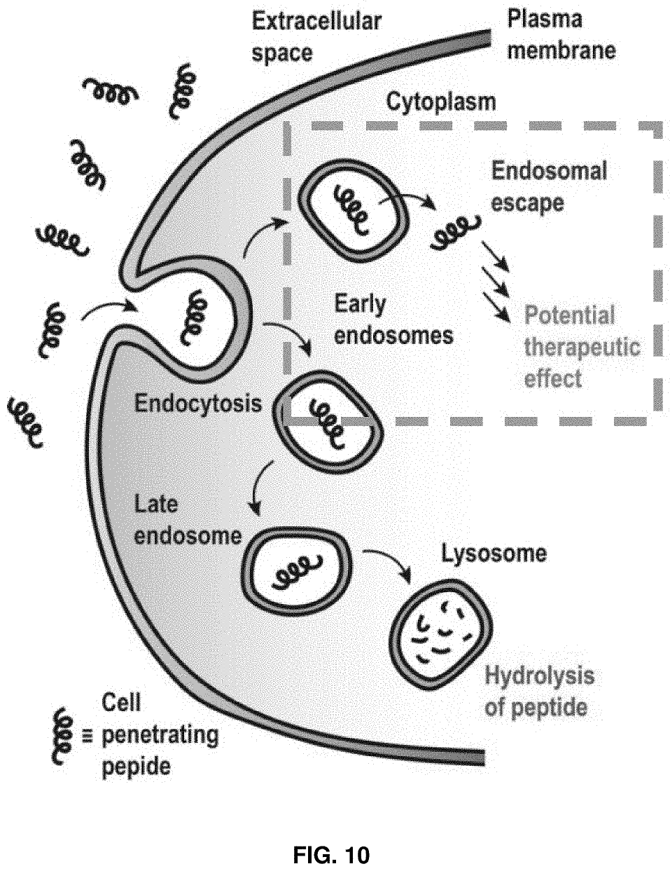

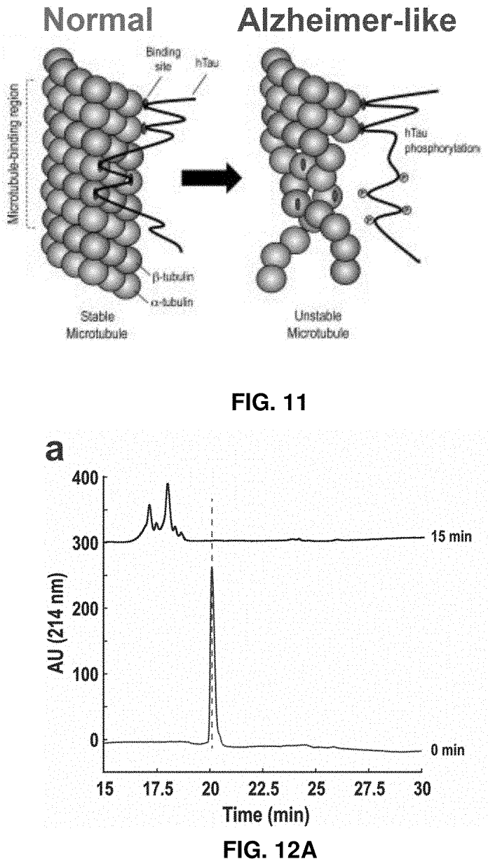

The purity of the peptide products was evaluated by analytical reversed-phase HPLC. Briefly, purified peptides were resuspended in 100% water at a final concentration of 2.5 μM and were injected across a reversed-phase analytical C18 column (Grace, 5 μm, 50×2.1 mm) and eluted within 20 minutes using a linear gradient of 5-95% solvent B (0.1% TFA in ACN) over solvent A (0.1% TFA in water). All peptides described herein were purified to >95% as determined by product peak integration of analytical HPLC spectra (FIG. 8A). Analytical HPLC data were acquired using OpenLab CDS ChemStation Software v1.06 (Agilent) and processed using KaleidaGraph v4.5 (Synergy Software).

The identity of the products was confirmed using electrospray ionization (ESI) mass spectrometry (FIG. 8B; FIG. 9-Table 1) using a Thermo Scientific Q Exactive Plus Hybrid Quadrupole-Orbitrap Mass Spectrometer in the m/z range of 500-2200. To perform the MS analysis, peptides were suspended in 500 μL 10% ACN in water and directly injected at a speed of 10 μL/min. All mass data were processed using Xcalibur v3.0 (Thermo) and MagTran v1.0 deconvolution software (Amgen, Thousand Oaks, Calif.).

Cleavage of the Fluorescently Labeled Peptides

After completing the synthesis, the peptide was cleaved from the resin by adding a mixture composed of 88% TFA, 5% Phenol, 2% Triisopropylsilane, and 5% H₂O (v/v) to the resin-bound peptide. Then, the reaction vessel containing the peptide was incubated in the microwave at 38° C. for 30 minutes. Following that, the peptide was precipitated in cold diethyl ether, pelleted by centrifugation, and re-suspended in 2-3 mL of 15% ACN (v/v) in water. This suspension was then frozen under −80° C. and lyophilized to dryness.

Plasmid Transformation

Plasmid coding for full-length human MARK2 were obtained from the DNASU Plasmid Repository (Tempe, Ariz.) in transformed phage resistant DH5-a TI E. coli bacterial cells (Clone ID: NC110760). MARK2 was supplied in a pMCSG7 vector, which adds an N-terminal poly-histidine tag (His₆) [SEQ ID NO: 4] to MARK2 to facilitate purification. Plasmids were isolated from DH5-α cells using a plasmid purification kit (Qiagen, Valencia, Calif.) and transformed into BL21(DE3) competent cells (Agilent, Santa Clara, Calif.) as described by to the manufacturer's instructions. After transformation, the cells were kept under −80° C. as glycerol stocks. Plasmid coding for human tau K18 was a generous gift from Professor Kevin G. Moffat, University of Warwick (Coventry, UK). hTau K18 was supplied in a pProEx-HTa-Myc-K18 vector, which adds a poly-histidine tag at the N-terminus of tau K18 Immediately upon arrival in our laboratory, the pProEx-HTa-Myc-K18 plasmids were transformed into Rosetta2 DE3 competent cells (Novagen, Darmstadt, Germany) as described by to the manufacturer's instructions. After transformation, the cells were kept under −80° C. as glycerol stocks.

Protein Expression and Purification

His-tagged MARK2 protein was expressed and purified from BL21(DE3) competent cells. Briefly, bacterial cells were inoculated overnight at 37° C. in LB broth supplemented with ampicillin with vigorous shaking at 225 rpm. The growth of the bacteria was measured and monitored until reaching 0.6 OD600. Expression of MARK2 was induced by the addition of IPTG at a final concentration of 1 mM. The induction was performed at 16° C. with shaking for 18 h. Following induction, the cultures were pelleted by centrifugation at 4° C. and frozen under −80° C. until further use. The pellet was re-suspended in buffer A (50 mM Tris-HCl, pH 7.5, 200 mM NaCl, 50 mM imidazole, 5 mM ChAPS, 2 mM benzamidine HCl, 1 mM β-mercaptoethanol, 1 mM PMSF), and was sonicated on ice 3× (10 seconds on, 1 minute off), and centrifuged 2× at 17,000 rpm for 30 minutes each at 4° C. The cleared supernatant was loaded onto an Ni-NTA column and incubated for 1 hour at 4° C. with shaking. Following incubation, unbound protiens were washed from the column with buffer A. MARK2 proteins were eluted with buffer B (50 mM Tris-HCl, 200 mM NaCl, 500 mM imidazole, 5 mM CHAPS, 2 mM benzamidine HCl, 1 mM β-mercaptoethanol, 1 mM PMSF, pH 7.5). Proteins from the washed and the eluted fractions were analyzed by SDS-PAGE (FIG. 5A). The volumes of the eluted fractions containing MARK2 were combined and dialyzed 2× in buffer (50 mM Tris-HCl, 5 mM MgCl₂, 2 mM EGTA, 0.5 mM DTT, 0.5 mM benzamidine HCl, 0.5 mM PMSF, pH 8.0). Following dialysis, protein was concentrated to between 10-20 μM, flash frozen and stored at −80° C. until further use. hTau-K18 with an N-terminal poly His-Tag was expressed and purified from Rosetta2 DE3 chemically competent cells. Briefly, cells were cultured at 37° C. in 1 L of LB broth with vigorous shaking at 180 rpm for 18 hours. Bacterial growth was monitored until reaching 0.6 OD600. Then, IPTG was added at a final concentration of 0.5 mM to induce expression of hTau-K18. The induction was performed at 37° C. for 1 hr with shaking (200 rpm). Following induction, the cultures was centrifuged 2× at 4° C. at 3500 rpm for 20 mm After centrifugation, the supernatant was removed and the pellet and was resuspended in 5 ml of 50 mM NaH₂PO₄—H₂O, pH 7.5. The pellet was stored at −80° C. until further use. Prior to purification, the lysate was heated to 42° C. for 10 minutes to gently thaw. Then, the protease inhibitor cocktail (Pierce mini tablet EDTA-Free, 1 tablet/˜50 ml lysate), 5 ml of 1×B-PER bacterial protein extraction reagent, and DNAse I was added to the lysate. The mixture was left at RT for 1 h, sonicated at 70% power for 1 mm, and centrifuged at 4° C. for 45 mm at 17,000 rpm. The supernatant was filtered through a cold 0.22 inn filter tube. Purification started with washing a Ni-NTA agarose column with buffer C (50 mM NaH₂PO₄, 500 mM NaCl, 10 mM imidazole, pH 7.0). Then, the clear lysate containing hTau-K18 was added to the washed column for 1 h at 4° C. with shaking. After collecting the crude extract, the column was washed with buffer D (50 mM NaH₂PO₄, 500 mM NaCl, 25 mM imidazole, pH 7.0). hTau-K18 was then eluted with buffer E (50 mM NaH₂PO₄, 500 mM 15 NaCl, 500 mM imidazole, pH 7.0). The protein was dialyzed 3× against dialysis buffer F (50 mM Tris HCl, 100 mM NaCl, pH 7.5). Following dialysis, protein was concentrated to between 10-20 μM, flash frozen, and stored under −80° C. until further use. Washing and elution fractions collected during tau K18 purification were separated on a 15% polyacrylamide gel by SDS-PAGE (FIG. 5B). Fractions containing purified protein were combined and dialyzed three times against buffer F (50 mM Tris-HCl, 100 mM NaCl, pH 7.5). Following dialysis, the protein was concentrated, flash frozen and stored under −80° C. until further use.

Circular Dichroism Analysis

The structure of His₆-MARK2 (“His₆” disclosed as [SEQ ID NO: 4]) was analyzed by wavelength-dependent circular dichroism (CD) spectropolarimetry. To perform this analysis, MARK2 was diluted to a final concentration of 5 μM in buffer (50 mM Tris-HCl, 5 mM MgCl₂, 2 mM EGTA, 0.5 mM DTT, 0.5 mM benzamidine HCl, 0.5 mM PMSF, pH 8.0). CD spectra were recorded from 245 nm to 195 nm at 25° C. (FIG. 5C). In order to assess the thermal stability of MARK2 in aqueous solution, MARK2 was diluted to a final concentration of 5 μM in the same buffer used to obtain CD spectra and monitored the temperature-dependence of the CD signal at 220 nm from 5 to 95° C. Melting temperature (Tₘ) was calculated by taking the first derivative of melting curve and determining the maximum value of the slope. The Tₘ. of MARK2 was found to be 61.1° C. Figures were designed using KaleidaGraph version 4.5 (Synergy Software) as described. The solution-phase structure of His-tagged hTau-K18 was analyzed similarly. Briefly, hTau-K18 was diluted to a final concentration of 10 μM in a buffer containing 10 mM NaHPO₄ H₂O, pH 7.4. CD spectra were recorded from 190 to 280 nm at 25° C. (FIG. 5D).

Western Blot Analysis for Observing the Inhibition of MARK2 by tR1 Peptide Using In Vitro Phosphorylation Assays

Phosphorylation assays were performed in 30 μL phosphorylation buffer (50 mM Tris-HCl, 5 mM MgCl₂, 2 mM EGTA, 0.5 mM DTT, 0.5 mM benzamidine HCl, 0.5 mM PMSF, pH 8.0) and depending on the experimental conditions. 100 μM ATP, 10 μM hTau K18, 10 nM MARK2, and 10 μM tR1 were added to the reaction mixture. The reaction was carried out for 1 hour at 32° C. The reaction was terminated by the addition of SDS sample buffer containing 84 mM Tris (pH 6.8), 20% glycerol, 4.6% SDS, 10% 2-mercaptoethanol, and 0.004% bromphenol blue, and then boiling for 3 mm at 95° C. The proteins were separated by SDS-PAGE on a 15% polyacrylamide gel. The proteins were then transferred to PVDF membranes for 2 hours at room temperature for western blot. After the transfer, the membrane was blocked overnight at 4° C. with 5% (w/v) nonfat dry milk in 1×TBS-Tween 20 (TBS-T), washed 3× with fresh TBS-T, and blotted with the antibodies specific for phospho-Tau Ser262 for 2 hours at room temperature. Unbound antibodies were washed from the membrane 3× using fresh TBS-T, before incubating with the secondary antibody Goat α-rabbit IgG (HRP) (1:5000 dilution was suspended in 5% milk in TBS-T) for 2 hr at RT. The membrane was washed using method as described above before signal detection using enhanced chemiluminescence (ECL) reagent. Images were acquired by a Bio-Rad Gel Doc EZ imager and the band intensity was quantified using Image Lab Software c5.2.1 (Bio-Rad).

Antibody-Based Fluorescence Polarization Assay

Analysis of the activity of MARK2 proteins was performed in 60-μL phosphorylation buffer (50 mM Tris-HCl, pH 8.0, 5 mM MgCl₂, 2 mM EGTA, 0.5 mM DTT, 0.5 mM benzamidine HCl, 0.5 mM PMSF) containing 100 μM ATP, 10 μM of ^{Flu}tR1, and 10 nM MARK2. The reaction mixture was incubated at 32° C. for 1 hour. Following incubation, 3 μg of the primary antibody α-phosho-Tau Ser262 was added to the reaction to detect phosphorylated R1 peptide. The reaction of the antibody with the phosphorylated ^{Flu}tR1 was allowed to proceed for 1 hour at room temperature. Fluorescence polarization was then measured using a SpectraMax M5e multi-mode plate reader (Molecular Devices, Sunnyvale, Calif.) in 384-well plates (#3575, Corning, Corning, N.Y.). The excitation wavelength used was 498 nm, and the emission wavelength was 525 nm (FIG. 6D).

Primary Culture of Rat Cortical Neurons

The care and use of animals adhered to the American Physiological Society's Guiding Principles in the Care and Use of Animals. All experimental procedures were approved by the Ohio University Institutional Animal Care and Use Committee. Brain dissection of embryonic (E-18) Sprague Dawley rat (Envigo) was performed as described previously. Dissociated cells were then plated onto 24 well culture plates (ThemoFisher 12-556-006) containing sterilized 12 mm circle no. 1 glass coverslips (ThermoFisher 12-545-80) coated with 0.05% polyethylenimine (polyE) (50% solution, Sigma-Aldrich P3143) in pH 8.2 borate buffer for the immunofluorescence. Dissociated cells were added to polyE coated glass coverslips in 24-well culture plates or polyE coated 24-well culture plates for the MTT assay or added to polyE coated six-well culture plates (Falcon 08-772-I B) for the western-blot. The primary cortical neurons were cultured in 0.5 mL minimum essential medium (MEM) (Corning Mediatech 50-010-PB) supplemented with 26 mM sodium bicarbonate, 55 mM glucose, 50 μg/mL gentamycin (Amresco E737), 20 mM KCl, 1 mM pyruvic acid (MP Biomedicals 102926), 2 mM L glutamine, and 10% (v/v) heat-inactivated fetal bovine serum (Atlas Biologicals FP-0050-A), in a 5% CO₂ incubator at 37° C. When cells attached to the coated surfaces after five hours, the conditioning media was switched to neurobasal (NB) (ThermoFisher Gibco 21103-049) supplemented with 0.5 mM L-glutamine, 2.8 μg/mL amphotericin B (Calbiochem 171375), and 2% (v/v) B27 supplement (ThermoFisher Gibco 17504-044).

Methyl-Thiazolyl-Diphenyl-Tetrazolium (MIT) Cell Viability Assay

High density rat primary cortical neurons (5-7 DIV) cultured in polyE coated 24-well plates were used for this assay. Culture media was switched to neurobasal without B27 supplement (NB-B27) before the addition of tR1 peptides. Then, 0 (control), 0.4, 1, 3, 10, 30, 100, or 300 μM tR1 peptides were added to the cells (three wells for each concentration). Cells were incubated with different concentrations of tR1 for 72 hours. For the MTT assay, 50 μg/mL MTT working solution was diluted from a 5 mg/mL MTT stock (water solution made from MTT powder, Alfa Aesar L11939) in NB-B27. After aspirating out the culture media, cells were incubated with 0.2 mL 50 μg/mL MTT working solution for one hour at 37° C. Then, the MTT solution was aspirated and replaced with 0.5 mL DMSO. The whole DMSO solution was transferred to a cuvette and read in the spectrophotometer (Cary UV 50, Varian) at 550 nm with subtraction of the reading at 650 nm wavelength.

Cell Uptake Assay of ^{Flu}tR1 Peptide

Cells were cultured for five to seven days in vitro (5-7 DIV) to ensure maturation of neuronal processes. Following incubation, the culture media was switched to fresh and warm NB+B27 with smaller volume (0.2 mL). And then, cells were incubated with 10 μM fluorescently labeled tR1 peptide, or co-incubated with 10 μM fluorescently labeled tR1 peptide and endosomal disruptor bafilomycin A1 (50 nM) (Sigma-Aldrich), for four hours under a humidified atmosphere (5% CO₂) at 37° C. Cells were then washed three times in warm pH 7.4 phosphate-buffered saline (PBS), and fixed in PBS containing 4% para-formaldehyde (20% solution, Electron Microscopy Service 15713) for eight minutes at room temperature. Finally, cells were washed 3× in PBS and mounted on glass slides (ThermoFisher 12-549-3) using 6.5 μl Anti-Fade/DAP reagent (ThermoFisher P36935) overnight in the dark.

Immunofluorescence

Cortical neurons (5-7 DIV), attached to polyE coated coverslips, were treated in conditions as described in the specific figure legends, and then washed 3× in warm pH 7.4 phosphate-buffered saline (PBS) and fixed in PBS containing 4% para-formaldehyde (20% solution, Electron Microscopy Service 15713) for eight minutes at room temperature. Fixed cells were then kept upright on a ceramic holder and rinsed three times through PBS before being permeabilized in 0.02% Triton X 100 (Sigma-Aldrich T8787) in PBS for one minute at room temperature, followed by three washes in PBS. Cells were then incubated with 1:1000 anti-tau (phospho 5262, rabbit polyclonal antibody) (Abcam, #ab5234), in 5% (w/v) non-fat milk/tris-buffered saline (TBS) supplemented with protease and phosphatase inhibitor cocktail (ThermoFisher 78440) for two hours at room temperature, and then incubated with 1:2000 Alexa 488 anti-rabbit secondary antibody (ThermoFisher A1 1034) in 5% non-fat milk/TB S supplemented with protease and phosphatase inhibitor cocktail for two hours at room temperature in the dark. The coverslips were finally washed three times through PBS and mounted on glass slides (ThermoFisher 12-549-3) using 6.5 μl Anti-Fade/DAPI reagent (ThermoFisher P36935) overnight in the dark.

Fluorescence Microscopy and Image Capture

Fluorescent imaging was examined using conventional epiflurosecence (Nikon, Diaphot 300) equipped with a Nikon Plan Apo 60×, 1.40 oil-immersion objective lens (used for immunofluorescence), and a Nikon Plan Apo 100×, 1.40 oil-immersion objective lens (used for fluorescent peptide uptake assay). For green fluorescence, the FITC-HYQ filter sets were used; for red fluorescence, TRITC Dil filter sets were used; for DAPI staining, the UV-2E/DAPI filter sets were used. Images were captured using a CCD camera (SPOT imaging).

Imaging Analysis

The fluorescent images were analyzed using the ImageJ program. For measuring the soma fluorescent intensity, neuronal area (soma and processes) was highlighted by applying the “Moments” automatic thresholding, and then the soma area was enclosed using the free selection tool, followed by measuring the mean intensity of the soma. Next, the background areas were determined and highlighted using the reversal selection tool to enclose the area outside neurons. All soma fluorescent intensities in this example were calculated with background subtraction.

Statistical Analysis

All data plots and statistical analyses were generated using GraphPad Prism 4.0 (GraphPad Software). Data are presented as mean±SEM. Where appropriate, data means were analyzed by one-way ANOVA followed by Tukey's multiple comparison test. Differences in the means were judged significant when P<0.05.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A composition comprising a tR1 peptide consisting of the amino acid sequence NVKSKIGSTENLK [SEQ ID NO: 1], or a variant thereof.

2. The composition of claim 1, wherein the variant has at least 61% sequence identity to the amino acid sequence of tR1.

3. The composition of claim 1, wherein the variant has at least 69% sequence identity to the amino acid sequence of tR1.

4. The composition of claim 1, wherein the variant has at least 76% sequence identity to the amino acid sequence of tR1.

5. The composition of claim 1, wherein the variant has at least 84% sequence identity to the amino acid sequence of tR1.

6. The composition of claim 1, wherein the variant has at least 92% sequence identity to the amino acid sequence of tR1.

7. The composition of claim 1, wherein the composition further comprises a pharmaceutically acceptable excipient, diluent, adjuvant, or carrier.

8. A method of inhibiting MARK2 function in a subject, the method comprising administering to a subject an effective amount of a tR1 peptide consisting of the amino acid sequence NVKSKIGSTENLK [SEQ ID NO: 1], or a variant thereof, to inhibit MARK2 function in the subject.

9. The method of claim 8, wherein the variant has at least 61% sequence identity to the amino acid sequence of tR1.

10. The method of claim 8, wherein the variant has at least 69% sequence identity to the amino acid sequence of tR1.

11. The method of claim 8, wherein the variant has at least 76% sequence identity to the amino acid sequence of tR1.

12. The method of claim 8, wherein the variant has at least 84% sequence identity to the amino acid sequence of tR1.

13. The method of claim 8, wherein the variant has at least 92% sequence identity to the amino acid sequence of tR1.

14. A method of inhibiting phosphorylation of tau Ser262 in primary cortical neurons, the method comprising administering to primary cortical neurons an effective amount of a tR1 peptide consisting of the amino acid sequence NVKSKIGSTENLK [SEQ ID NO: 1], or a variant thereof, to inhibit phosphorylation of tau Ser262 in the primary cortical neurons.

15. The method of claim 14, wherein the tR1 peptide does not inhibit GSK-3β-mediated phosphorylation of tau Thr231 in the primary cortical neurons.

16. The method of claim 14, wherein the tR1 peptide is internalized by the primary cortical neurons via endocytosis.

17. The method of claim 14, wherein the variant has at least 61% sequence identity to the amino acid sequence of tR1.

18. The method of claim 14, wherein the variant has at least 69% sequence identity to the amino acid sequence of tR1.

19. The method of claim 14, wherein the variant has at least 76% sequence identity to the amino acid sequence of tR1.

20. The method of claim 14, wherein the variant has at least 84% sequence identity to the amino acid sequence of tR1.

21. The method of claim 14, wherein the variant has at least 92% sequence identity to the amino acid sequence of tR1.

22. A method of inhibiting a MARK family protein in a subject, the method comprising administering to a subject an effective amount of a tR1 peptide consisting of the amino acid sequence NVKSKIGSTENLK [SEQ ID NO: 1], or a variant thereof, to inhibit a MARK family protein in the subject.

23. The method of claim 22, wherein the variant has at least 61% sequence identity to the amino acid sequence of tR1.

24. The method of claim 22, wherein the variant has at least 69% sequence identity to the amino acid sequence of tR1.

25. The method of claim 22, wherein the variant has at least 76% sequence identity to the amino acid sequence of tR1.

26. The method of claim 22, wherein the variant has at least 84% sequence identity to the amino acid sequence of tR1.

27. The method of claim 22, wherein the variant has at least 92% sequence identity to the amino acid sequence of tR1.

28. A method of treating, preventing, or ameliorating a neurodegenerative disease, the method comprising administering to a subject in need thereof an effective amount of a tR1 peptide consisting of the amino acid sequence NVKSKIGSTENLK [SEQ ID NO: 1], or a variant thereof, to treat, prevent, or ameliorate a neurodegenerative disease in the subject.

29. The method of claim 28, wherein the neurodegenerative disease is Alzheimer's disease or frontotemporal dementia.

30. The method of claim 28, wherein the variant has at least 61% sequence identity to the amino acid sequence of tR1.

31. The method of claim 28, wherein the variant has at least 69% sequence identity to the amino acid sequence of tR1.

32. The method of claim 28, wherein the variant has at least 76% sequence identity to the amino acid sequence of tR1.

33. The method of claim 28, wherein the variant has at least 84% sequence identity to the amino acid sequence of tR1.

34. The method of claim 28, wherein the variant has at least 92% sequence identity to the amino acid sequence of tR1.

35. Use of a synthetic peptide to inhibit MARK2-mediated tau phosphorylation.

36. The use of claim 35, wherein the synthetic peptide consists of the amino acid sequence NVKSKIGSTENLK [SEQ ID NO: 1], or a variant thereof.

37. The use of claim 35, wherein the synthetic peptide mimics the tau R1 repeat domain.

38. The use of claim 35, wherein the variant has at least 61% sequence identity to the amino acid sequence of tR1.

39. The use of claim 35, wherein the variant has at least 69% sequence identity to the amino acid sequence of tR1.

40. The use of claim 35, wherein the variant has at least 76% sequence identity to the amino acid sequence of tR1.

41. The use of claim 35, wherein the variant has at least 84% sequence identity to the amino acid sequence of tR1.

42. The use of claim 35, wherein the variant has at least 92% sequence identity to the amino acid sequence of tR1.

43. Use of a synthetic peptide to inhibit a MARK family protein.

44. The use of claim 43, wherein the synthetic peptide consists of the amino acid sequence NVKSKIGSTENLK [SEQ ID NO: 1], or a variant thereof.

45. The use of claim 43, wherein the synthetic peptide mimics the tau R1 repeat domain.

46. The use of claim 43, wherein the variant has at least 61% sequence identity to the amino acid sequence of tR1.

47. The use of claim 43, wherein the variant has at least 69% sequence identity to the amino acid sequence of tR1.

48. The use of claim 43, wherein the variant has at least 76% sequence identity to the amino acid sequence of tR1.

49. The use of claim 43, wherein the variant has at least 84% sequence identity to the amino acid sequence of tR1.

50. The use of claim 43, wherein the variant has at least 92% sequence identity to the amino acid sequence of tR1.


SEQUENCE LISTING (4 sequences)

SEQ ID NO: 1 - protein, 13 aa, Artificial Sequence
  Description of Artificial Sequence Synthetic peptide
NVKSKIGSTENLK

SEQ ID NO: 2 - protein, 5 aa, Homo sapiens
ICVGS

SEQ ID NO: 3 - protein, 4 aa, Homo sapiens
KIGS

SEQ ID NO: 4 - protein, 6 aa, Artificial Sequence
  Description of Artificial Sequence Synthetic 6xHis tag
HHHHHH